(19) United States
(12) Patent Application Publication
(10) Pub. No.: US 2024/0225700 A1
(43) Pub. Date: Jul. 11, 2024

(54) EXTRUDED LEAN MAGNESIUM-CALCIUM ALLOYS

(71) Applicant: ETH ZÜRICH, Zürich (CH)

(72) Inventors: Leopold BERGER, Zürich (CH); Jörg F. LÖFFLER, Greifensee (CH); Samuel MONTIBELLER, Unterkulm (CH)

(73) Assignee: ETH ZÜRICH, Zürich (CH)

(21) Appl. No.: 18/576,883

(22) PCT Filed: Jul. 8, 2022
(86) PCT No.: PCT/EP2022/069062

§ 371 Date: Jan. 5, 2024

(30) Foreign Application Priority Data
Jul. 9, 2021 (EP) 21184885.8

(51) Int. Cl. A61B 17/68; A61B 17/00; C22C 1/02; C22C 23/00; C22F 1/06

(52) U.S. Cl. CPC A61B 17/68; C22C 1/02; C22C 23/00; C22F 1/06; A61B 2017/00004; A61B 2017/00526

(57) ABSTRACT

A method of producing an alloy including magnesium and calcium, preferably an implantable medical device having magnesium and calcium, includes the steps of generating a billet including magnesium and calcium, and extruding the billet. The billet is extruded at least once at an extrusion temperature in the range of 250° C. to 450° C. and at a ram speed in the range of 0.01 mm/s to 1 mm/s and at an extrusion ratio in the range of 20 to 150 and preferably at an extrusion ratio in the range of 35 to 150.

### TECHNICAL FIELD

The present invention relates to a method of producing an alloy comprising magnesium and calcium according to claim 1, to an alloy comprising magnesium and calcium according to claim 12, to the use of such an alloy as an implantable medical device according to claim 15, and to an implantable medical device comprising or consisting of such an alloy according to claim 16, respectively.

### PRIOR ART

Musculoskeletal trauma accounts for 85% of all traumatic injuries worldwide, with fractures being the most common type. Thankfully, fracture healing is one of the few truly regenerative processes that lead to a complete restoration of the original tissue state. While implants, such as screws, plates, nails or wires, are typically made of stainless steel or titanium alloys and are commonly used for the surgical treatment of bone fractures, there is a persistent issue that remains with regards to implant removal after the desired fixation has been achieved. This applies not only to fracture fixation implants but also to various other implants currently used, such as vascular scaffolds, surgical clips, suture anchors, screws for the fixation of ruptured anterior cruciate ligaments, membranes for guided bone restoration, porous bone-scaffolds, etc. Implant-related complications such as pain, implant failure, dislocations, peri-implantitis, necrosis, protruding implant, growth distortions in children, functional impairment or cosmetics cannot be ignored, and generate up to 30% of complications, making it statistically significant enough to medically advise removal surgery in many cases (Vos & Verhofstad, 2013). These surgeries are expensive, imply more hospital time and are an economic and social burden on the patients.

In the last years, a promising new class of biodegradable implant materials—magnesium and its alloys—emerged and has attracted considerable attention.

Major hurdles that have to be overcome on the path towards clinical applicability of magnesium-based implants are the low mechanical strength and ductility of pure magnesium and the often far too rapid degradation rate and accompanied hydrogen gas evolution of magnesium alloys when subjected to degradation by aqueous solutions. Hydrogen gas is a reaction product of Mg and water (1 mole Mg leads to 1 mole H₂) that diffuses safely through tissues when evolving moderately, but can cause large, macroscopic cavities in bone and soft tissue when the implanted material degrades too quickly.

In order to address these issues, numerous approaches have been made to increase the degradation resistance of magnesium with various alloying strategies. One proposed solution are magnesium alloys containing yttrium and/or rare-earth elements, such as neodymium or gadolinium, and/or zirconium. Originally developed for industrial high-performance light-weight applications these alloys also have high degradation resistance and excellent mechanical properties. While performing mainly satisfying in pre-clinical clinical tests, recent studies observed that rare-earth elements can negatively influence apoptosis and the viability of immune cells, and that rare-earth elements, originally non-abundant in the human body, accumulate in bones and organs (Jin, Wu, Yuan, & Chen, 2018; Myrissa et al., 2017). It is therefore strongly advisable to develop rare-earth-element-free magnesium alloys specifically for biomedical applications.

The currently most promising alternatives to rare-earth-element-comprising magnesium alloys are magnesium-zinc-calcium alloys with zinc and calcium being essential nutrients and therefore intrinsically biocompatible. Although, mechanically strong and biocompatible, alloy versions with relatively high Ca and/or Zn content typically exhibit a degradation rate and accompanied hydrogen evolution that is either clearly too high or rather close to the tolerable limit (Kraus et al., 2012).

Therefore, further efforts are needed to reduce degradation rate by simultaneously keeping the favorable mechanical and biocompatible properties. In particular, so-called high-strength low alloy Mg—Zn—Ca alloys were developed and exhibited promising properties. Tensile yield strength of about 240 MPa with simultaneous high ductility, at an elongation at fracture of about 30%, were achieved (Hofstetter et al., 2015). Elongation at fracture, as a measure of ductility, is typically determined by tensile testing and defined as the permanent elongation of the length of the parallel portion of the test piece after fracture of the test specimen, expressed as a percentage of the original length of the parallel portion of the test piece.

Additionally to the above described developments, a vast number of other alloy combinations were investigated in recent years but deemed unsuitable for biomedical applications either due to concerns about biocompatibility, with most notable Mg—Al and Mg-rare-earth-element alloys, or unfavorable electrochemical properties leading to extensive degradation rates, for example Mg—Zn or Mg—Zn—Zr (Han et al., 2019).

The above-mentioned alloying element Zn is commonly considered as an important requirement to achieve strong and ductile aluminum-free and rare-earth-element-free magnesium alloys as Zn is known to add strongly to solid-solution strengthening and foster the activation of so-called c+a non-basal slip (Basu, Chen, Wheeler, Schäublin, & Löffler, 2021). This activation of an additional slip-system can improve ductility of the resulting material in a very significant way. Ductility, the ability of plastic deformation, is a pre-requisite for the application as degradable bone implant in many cases, since the envisioned biodegradable implants require often an inter-surgery fitting to the fractured bone by bending and thereby plastic deformation. In other conceivable implant applications, such as surgical clips, membranes, wires, plates, or vascular scaffolds the ability to plastically deform is also of crucial importance.

On the contrary, recent research discovered that the above-mentioned alloying element Zn is a significant factor in the unwanted acceleration of corrosion of Mg—Zn—Ca alloys. The reason is the relatively large difference in electrochemical potential to the Mg matrix, with Zn being much nobler than Mg, causing increased corrosion activity. Additionally, Zn and Ca might form particles of ternary Mg—Zn—Ca intermetallic phases, being again nobler than Mg and thus cathodic to the Mg matrix, thereby causing a strongly detrimental effect of increased degradation rate and hydrogen evolution in vivo. It was found that these intermetallic phases even tend to de-alloy when subjected to corrosive media, further increasing their cathodic activity and thereby leading to an additional unwanted acceleration of corrosion. Also re-deposition of Zn onto the alloy surface has been observed, which is again detrimental to the corrosion resistance of Zn-comprising magnesium alloys (Cihova, Schmutz, Schäublin, & Löffler, 2019).

This is a major drawback as lowest possible corrosion in vivo is desired for many potential implant applications and it would therefore be very beneficial to reach the same or better biocompatibility, the same or better mechanical properties and at the same time better electrochemical properties than those of Mg—Zn—Ca alloys, without using the alloying element Zn.

Although Zn as an additional element was up to date considered as a very beneficial alloying element and an important contribution to promote c+a pyramidal slip for a technically required level of ductility (Basu et al., 2021), the sole addition of Ca to Mg is known to the art and used in cast alloys with positive effects on grain refinement and mechanical strength. However, these alloys are also known especially for being very brittle and therefore no option for a load-bearing degradable implant. Zn-free Mg—Ca alloys have also been subjected to hot-deformation processing, with promising, although not excellent results. (Pan et al., 2019) describes the hot extrusion of Mg-0.1 wt % Ca and Mg-1 wt % Ca. In general, throughout the present document “wt %” is used, as it is typically used for the quantification of alloy contents and impurities, and refers to the “weight of an alloying element or impurity element per total weight of the alloy expressed as a fraction of 100”. A tensile yield strength of up to 377 MPa, at a very low elongation at fracture of 2%, or a mediocre elongation at fracture of 18%, at a low tensile yield strength of about 219 MPa were achieved. Thus, for successful application in cases where high strength is needed, ductility is too low and for applications in cases where high ductility is desired, the here achieved ductility might not be enough and at a rather low mechanical strength level.

Extruded Mg—Ca alloys with a Ca content of 0.8 wt % as base material for biodegradable implants were described in (Krause et al., 2010). Results provided on degradation behaviour and degradation speed in vivo leads to the educated guess that the used materials were not of high purity and/or not ideally processed, therefore leading to a degradation rate that was deemed by the study authors as being too high for application. The authors do not provide specific values on strength and ductility, but state that their employed implant shows “shows an insufficient initial strength”, another indicator of a strong need for improvement, if such a material would be deployed as an implant. Whenever provided in these examples, Ca was introduced to the magnesium melt by using a Mg—Ca master alloy.

WO 2014/001321 A1 describes a magnesium alloy consisting of magnesium and calcium. The material is subjected to multiple processing steps such as multiple extrusions and multiple heat treatments before and after extrusions. As described, apparently multiple extrusions are needed to achieve these results. Multiple extrusions are economically strongly unfavorable, due to the prolonged processing time and since only a small part of the firstly extruded material can be subjected to the second (or third, . . . ) extrusion at a time. In addition, WO 2014/001321 A1 is silent on most of the parameters used with respect to the extrusion processes, and completely silent on further mechanical properties such as the ductility and/or strength of such alloys, and on related electrochemical properties of such alloys. Therefore, achieving improved mechanical and electrochemical properties by a less elaborate process with the possibility of tailoring mechanical properties by a targeted variation of extrusion parameters would be a significant improvement.

CN101015711B describes binary Mg—Ca alloys with examples provided for different compositions. The alloy of the lowest reported Ca-content, Mg-1 wt % Ca is reported to be prepared by hot rolling and exhibits a relatively low tensile yield strength of about 170 MPa at less than 4% of elongation at fracture. Especially the low value of elongation at fracture at additionally low strength make this material unsuitable for the majority of applications.

CN102312144A claims binary Mg—Ca alloys with a Ca content equal to or above 0.5 wt %. It describes a relatively small grain size (above 3 micrometers) and also describes hot extrusion as potential method to achieve this grain size. A Mg-1 wt % Ca alloy is reported after again multiple extrusion steps with an ultimate tensile strength reaching 360 MPa and elongation at fracture of 15% at a grain size of 3-5 micrometers. Extrusion parameters are provided for the first extrusion step only with an extrusion speed of 80 mm/s and an extrusion ratio of 10. Again, achieving similar or better mechanical results with just a single extrusion step would be a significant improvement.

Hence, in summary it can be said that the known methods or alloys in the field of the invention suffer from several drawbacks such as insufficient mechanical properties, in particular ultimate tensile strength and elongation at fracture, and/or are obtainable only in an elaborate manufacturing process.

### REFERENCES

Basu, I., Chen, M., Wheeler, J., Schäublin, R. E., & Löffler, J. F. (2021). Stacking-fault mediated plasticity and strengthening in lean, rare-earth free magnesium alloys. Acta Materialia, 211, 116877. https://doi.org/10.1016/j.actamat.2021.116877
Cihova, M., Schmutz, P., Schäublin, R., & Löffler, J. F. (2019). Biocorrosion Zoomed In: Evidence for Dealloying of Nanometric Intermetallic Particles in Magnesium Alloys. Advanced Materials, 31(42), el903080. https://doi.org/i0.1002/adma.201903080
Han, H. S., Loffredo, S., Jun, I., Edwards, J., Kim, Y. C., Seok, H. K., . . . Glyn-Jones, S. (2019). Current status and outlook on the clinical translation of biodegradable metals. Materials Today, 23, 57-71. https://doi.org/10.1016/j.mattod.2018.05.018
Hofstetter, J., Ruedi, S., Baumgartner, I., Kilian, H., Mingler, B., Povoden-Karadeniz, E., . . . Löffler, J. F. (2015). Processing and microstructure-property relations of high-strength low-alloy (HSLA) Mg—Zn—Ca alloys. Acta Materialia, 98, 423-432. https://doi.org/10.1016/j.actamat.2015.07.021
Jin, L., Wu, J., Yuan, G., & Chen, T. (2018). In vitro study of the inflammatory cells response to biodegradable Mg-based alloy extract. PLoS ONE, 13(3), 1-15. https://doi.org/10.1371/journal.pone.0193276
Kraus, T., Fischerauer, S. F., Hänzi, A. C., Uggowitzer, P. J., Löffler, J. F., & Weinberg, A.-M. (2012). Magnesium alloys for temporary implants in osteosynthesis: In vivo studies of their degradation and interaction with bone. Acta Biomaterialia, 8(3), 1230-1238. https://doi.org/10.1016/j.actbio.2011.11.008
Krause, A., Von Der Höh, N., Bormann, D., Krause, C., Bach, F. W., Windhagen, H., & Meyer-Lindenberg, A. (2010). Degradation behaviour and mechanical properties of magnesium implants in rabbit tibiae. Journal of Materials Science, 45(3), 624-632. https://doi.org/i0.1007/s10853-009-3936-3
Myrissa, A., Braeuer, S., Martinelli, E., Willumeit-Römer, R., Goessler, W., & Weinberg, A.-M. (2017). Gadolinium accumulation in organs of Sprague-Dawley® rats after implantation of a biodegradable magnesium-gadolinium alloy. Acta Biomaterialia, 48, 521-529. https://doi.org/10.1016/j.actbio.2016.11.024
Pan, H., Yang, C., Yang, Y., Dai, Y., Zhou, D., Chai, L., . . . Qin, G. (2019). Ultra-fine grain size and exceptionally high strength in dilute Mg—Ca alloys achieved by conventional one-step extrusion. Materials Letters, 237, 65-68. https://doi.org/10.1016/j.matlet.2018.11.080
Vos, D. I., & Verhofstad, M. H. J. (2013). Indications for implant removal after fracture healing: A review of the literature. European Journal of Trauma and Emergency Surgery, 39(4), 327-337. https://doi.org/10.1007/s00068-013-0283-5

### SUMMARY OF THE INVENTION

It is an object of the present invention to provide a method of producing an alloy of improved and/or tailored mechanical properties that is less elaborate.

This object is achieved with the method according to claim 1. In particular, a method of producing an alloy comprising magnesium and calcium, preferably a method of producing an implantable medical device comprising magnesium and calcium, is provided, wherein the method comprises the steps of i) generating a billet comprising magnesium and calcium, and ii) extruding the billet. The billet is extruded at least once at an extrusion temperature in the range of 250° C. to 450° C. and at a ram speed in the range of 0.01 mm/s to 1 mm/s and at an extrusion ratio in the range of 20 to 150 and particularly preferably at an extrusion ratio in the range of 35 to 150.

Extrusion is well-known in the art and corresponds to a pressure-assisted forming process with pressure being exerted on a billet that is comprised in a container. The pressure is applied on the billet by a ram that is travelling through the container. The expression “billet” is well-known in the art as well and refers to the material being subject to the extrusion, for instance being pushed through a die of an extrusion equipment by the ram. Here, the billet comprising magnesium and calcium means that material comprising magnesium and calcium is extruded in an extrusion equipment. Said billet comprising magnesium and calcium preferably corresponds to a magnesium-calcium alloy, also referred to as a Mg—Ca-alloy.

The alloy in the form of the billet shall not be confused with the alloy being obtained after extrusion of the billet. In fact, the alloy obtained after the extrusion of the billet and, if applicable, after any further method steps such as a heat treatment (see further below) can be seen as a final alloy or target alloy, i.e. an alloy that comprises the improved and/or tailored mechanical properties, in particular the improved and/or tailored ultimate tensile strength and elongation at fracture. For the sake of clarity, the alloy in the form of the billet is hereinafter referred to as intermediate alloy.

The billet being extruded at an extrusion temperature in the range of 250° C. to 450° C. means that the billet or Mg—Ca-alloy, i.e. the intermediate alloy, is held at a temperature in said range while it is extruded, for example being pushed through the die of an extrusion equipment.

In fact, and as will be explained in greater detail below, prior to the extrusion the billet is preferably pre-heated until it reaches the said extrusion temperature. In parallel the extrusion die and the extrusion device, in particular the extrusion container, are preferably pre-heated to the extrusion temperature as well. Hence, it is particularly preferred to perform the extrusion when all three components, i.e. the billet, the die and the extrusion device, are at the extrusion temperature, or in a close range to the extrusion temperature. Once this state is reached the billet is preferably inserted into the extrusion device and the extrusion process is performed. Hence, the extrusion according to the invention preferably corresponds to a hot extrusion and the extrusion device preferably is a hot-extrusion device.

The expression “ram speed” is also well-known in the art and refers to the speed at which the ram is travelling through the extrusion container during the extrusion process.

The expression “extrusion ratio” is also well-known in the art and corresponds to the ratio between an initial cross-sectional area of the extrusion container and a final cross-sectional area of the billet after extrusion. The extrusion ratio R can be calculated as follows:

$$R = \frac{{A}_{0}}{{A}_{f}} ,$$

wherein A₀ corresponds to the initial cross-sectional area of the extrusion container and A_{f} corresponds to the final cross-sectional area of the billet after extrusion.

Thus, and as an example, the extrusion ratio of 75 was obtained by an extrusion container comprising an initial cross-sectional area of 2123.7 square millimeter that was used to extrude a billet of originally a slightly smaller cross-sectional area through a die comprising a cross-sectional area of about 28.3 square millimeter, so as to result in a billet after extrusion having a final cross-sectional area of about 28.3 square millimeter. It should be noted that the extrusion ratio designation “R” used here is identical to the extrusion ratio designation “R:1” occasionally used in the art.

The extrusion preferably is an indirect extrusion, wherein only the ram is moving with respect to the extrusion container while the billet is kept stationary with respect to the extrusion container.

It should be noted that the method steps, in particular the step of extrusion, can be performed at least once or only once. In fact, and unlike in the prior art, improved material properties were already achieved in the method according to the invention by performing only a single extrusion step.

The inventors have found out that optimal extrusion parameters, in particular the extrusion temperature, the ram speed and the extrusion ratio, enable the targeted production of the alloy comprising not only improved material properties, in particular improved ultimate tensile strength and improved elongation at fracture and for instance a very high ductility, but also targeted material properties spanning a very wide range. Furthermore, it is noted at this point that the indicated extrusion ration, in particular the preferred extrusion ratio in the range of 35 to 150 mentioned above, is associated with several advantages. Namely, from a materials science perspective it is associated with a greater degree of plastic deformation during processing and consequently increases the grain fining effect and results in a higher strength (so-called Hall Petch strengthening). From an economic perspective it offers greater productivity. In particular, the work needed to produce the billet before extrusion is basically independent of its size. As such, a double extrusion ratio means a double-sized billet can be used for the same extrusion cross section. Consequently, the indicated preferred extrusion ratio results in roughly half labor costs per amount of extruded material.

All provided information and terminology with respect to tensile properties of materials are determined and to be understood according to the standard ISO 6892-1, determined at a strain rate between 0.0008 and 0.001 per second.

In fact, the extrusion parameters in the method according to the invention are preferably such that an alloy is produced having an ultimate tensile strength in the range of 100 MPa to 500 MPa, such as in the range of 150 MPa to 500 MPa or in the range of 150 MPa to 250 MPa, or in the range of 250 MPa to 300 MPa, or in the range of 150 MPa to 300 MPa, or in the range of 250 MPa to 350 MPa, or in the range of 380 MPa to 450 MPa. In other words, the alloy can have an ultimate tensile strength of more than 100 MPa, or of more than 150 MPa, or of more than 250 MPa, or of more than 300 MPa, or of more than 380 MPa.

Additionally or alternatively, the method according to the invention enables the production of the alloy having an elongation at fracture in the range of 2% to 50%, such as in the range of 2% to 40% or of 2% to 35%, or in the range of 2% to 8%, or in the range of 2% to 10%, or in the range of 8% to 15%, or in the range of 10% to 25%, or in the range of 25% to 40%, or in the range of 30% to 40%. In other words, the alloy can have an elongation of fracture of 2% or more, or of 10% or more, or of 20% or more, or of 25% or more, or of 35% or more.

To this end it is particularly preferred that the alloy has an ultimate tensile strength in the range of 100 MPa to 500 MPa and an elongation at fracture of 2% to 50%, more preferably an ultimate tensile strength in the range of 150 MPa to 500 MPa and an elongation at fracture of 2% to 40%.

Furthermore, the method according to the invention enables the production of an alloy having particular material properties, such as an alloy for high-strength applications, or an alloy for applications where a high plasticity or ductility is needed, i.e. an alloy having a high ductility, or an alloy having an optimized combination of strength and ductility.

For instance, an alloy for high-strength applications preferably has an ultimate tensile strength of 300 MPa or more and an elongation at fracture in the range of 2% to 15%. More preferably, such an alloy for high-strength applications has an ultimate tensile strength of 350 MPa or more and an elongation at fracture in the range of 2% to 10%.

The alloy having high strength is preferably produced in the method according to the invention by extruding the billet at least once at an extrusion temperature in the range of 300° C. to 400° C., and at a ram speed in the range of 0.05 mm/s to 0.2 mm/s, and at an extrusion ratio in the range of 50 to 100. To this end it is furthermore preferred that the alloy comprises between 0.25% by weight and 0.65% by weight of calcium based on the total weight of the alloy. A particularly preferred alloy having a high strength is preferably produced in the method according to the invention by extruding the billet at least once at an extrusion temperature in the range of 330° C. to 380° C., and at a ram speed in the range of 0.05 mm/s to 0.15 mm/s, and at an extrusion ratio in the range of 65 to 80. To this end it is furthermore preferred that the alloy comprises between 0.25% by weight and 0.65% by weight such as between 0.3% by weight and 0.65% by weight of calcium based on the total weight of the alloy.

An alloy having a high ductility or where high plasticity or ductility is needed preferably has an ultimate tensile strength of 150 MPa or more and an elongation at fracture of 20% or more. The alloy having high ductility is preferably produced in the method according to the invention by extruding the billet at least once at an extrusion temperature in the range of 360° C. to 430° C., and at a ram speed in the range of 0.15 mm/s to 0.6 mm/s, and at an extrusion ratio in the range of 50 to 100. To this end it is furthermore preferred that the alloy comprises between 0.3% by weight and 0.65% by weight of calcium based on the total weight of the alloy. A particularly preferred alloy having high ductility is preferably produced in the method according to the invention by extruding the billet at least once at an extrusion temperature in the range of 370° C. to 390° C., and at a ram speed in the range of 0.2 mm/s to 0.55 mm/s, and at an extrusion ratio in the range of 65 to 80. To this end it is furthermore preferred that the alloy comprises between 0.25% by weight and 0.65% by weight such as between 0.4% by weight and 0.65% by weight of calcium based on the total weight of the alloy.

An alloy having an optimized combination of strength and ductility preferably has an ultimate tensile strength in the range of 250 MPa to 350 MPa and an elongation at fracture in the range of 8% to 25%. The alloy having an optimized combination of strength and ductility is preferably produced in the method according to the invention by extruding the billet at least once at an extrusion temperature in the range of 330° C. to 400° C., and at a ram speed in the range of 0.15 mm/s to 0.5 mm/s, and at an extrusion ratio in the range of 50 to 100. To this end it is furthermore preferred that the alloy comprises between 0.25% by weight and 0.65% by weight of calcium based on the total weight of the alloy. A particularly preferred alloy having an optimized combination of strength and ductility is preferably produced in the method according to the invention by extruding the billet at least once at an extrusion temperature in the range of 330° C. to 360° C., and at a ram speed in the range of 0.2 mm/s to 0.3 mm/s, and at an extrusion ratio in the range of 65 to 80. To this end it is furthermore preferred that the alloy comprises between 0.25% by weight and 0.65% by weight such as between 0.25% by weight and 0.4% by weight of calcium based on the total weight of the alloy.

The production of alloys having particular mechanical properties is especially desirable in the field of implants, such as implants being used in orthopedic surgery such as in osteosynthesis and/or in dental applications. For instance, after a bone fracture it might be necessary to stabilize the fracture using osteosynthesis such as a plate and screws to fix the plate to the bone. The material requirements for these implants can be very different. For example, strong screws are desired for a good fixation of the screws in bones. In the case of the plate, on the other hand, it is often desirable for it to have a certain ductility. This is particularly important for younger patients who are still growing.

Hence, the present invention not only enables the production of an alloy having improved material properties, in particular a high ultimate tensile strength and a large elongation at fracture, but also to tailor the material properties for specific applications of the alloy.

The billet can furthermore comprise zirconium and/or hafnium. In other words, the method can comprise the step of generating a billet comprising magnesium, calcium and zirconium and/or hafnium, i.e. a Mg—Ca—Zr intermediate alloy, a Mg—Ca—Hf intermediate alloy or a Mg—Ca—Zr—Hf intermediate alloy can be produced. Consequently, the alloy produced in the method, i.e. the final alloy or target alloy, can comprise magnesium, calcium and additionally also zirconium and/or hafnium. Again, in other words, an alloy comprising or consisting of magnesium and calcium, i.e. a Mg—Ca alloy, can be produced. However, it is likewise conceivable that an alloy comprising or consisting of magnesium and calcium and zirconium, i.e. a Mg—Ca—Zr alloy, is produced. Likewise, it is conceivable that an alloy comprising or consisting of magnesium and calcium and hafnium, i.e. a Mg—Ca—Hf alloy, is produced. It is furthermore conceivable that an alloy comprising or consisting of magnesium and calcium and zirconium and hafnium, i.e. a Mg—Ca—Zr—Hf alloy, is produced.

The alloy preferably comprises between 0.15% by weight and 1.0% by weight of calcium based on the total weight of the alloy. More preferably, the alloy comprises between 0.25% by weight and 0.7% by weight of calcium based on the total weight of the alloy. For instance, the alloy can comprise between 0.3% by weight and 0.65% by weight of calcium based on the total weight of the alloy. Or, the alloy can comprise between 0.4% by weight and 0.65% by weight of calcium based on the total weight of the alloy. Or, the alloy can comprise between 0.25% by weight and 0.4% by weight of calcium based on the total weight of the alloy. Or, the alloy can comprise between 0.6% by weight and 1.0% by weight of calcium based on the total weight of the alloy.

The alloy preferably comprises an intermetallic phase of Mg₂Ca. It is furthermore preferred that the intermetallic phase of Mg₂Ca is present in a mole fraction in the range of 0% to 1.5%, more preferably in the range of 0.05% to 1.5%, even more preferably in the range of 0.1% to 1.5% and particularly preferably in the range of 0.2% to 1.5%. This intermetallic phase of Mg₂Ca preferably precipitates within the alloy during a period of elevated temperature prior to an extrusion step, beginning with small clusters that get bigger in size with time, eventually forming particles that also increase in size with time, but getting smaller in number. This period of elevated temperature can either be a separate heat treatment or happen during the necessary pre-heating prior to an extrusion process. Provided that the temperature is below the solvus temperature of this phase, its mole fraction as well as particle size and number density are controlled by the temperature-time profile during the mentioned period of elevated temperature.

The mole fraction of the intermetallic phase of Mg₂Ca is preferably determined using transmission electron microscopy (TEM). TEM micrographs allow to identify the Mg₂Ca intermetallic phase as particles distributed in the material and to determine the size of the particles by using image analysis software preferable according to ISO 13322-1. The term “size” preferably corresponds to the “mean area equivalent diameter” as defined in ISO 13322-1. Additionally, the area number density of the Mg₂Ca intermetallic particles, n_{A,Mg2Ca} can be obtained from TEM micrographs (by using image analysis software preferable according to ISO 13322-1). The particles' volume number density n_{V,Mg2Ca} is then computed by dividing the area number density by the average thickness of the measured region, t as determined using Electron Energy Loss Spectroscopy or by stereography:

$${n}_{V , Mg 2 Ca} = \frac{{n}_{A , Mg 2 Ca}}{t} ,$$

where n_{A,Mg2Ca} is the Mg₂Ca intermetallic particles' area number density and t is the average thickness of the measured region.

Multiplying the volume number density with the volume of an assumed representative spherical-shaped particle gives furthermore the volume fraction of Mg₂Ca as present in the alloy, ϕ_{Mg2Ca}. As diameter of this representative spherical-shaped particle the earlier experimentally determined mean area equivalent diameter of the Mg₂Ca particles is used. The volume fraction of Mg₂Ca as present in the alloy, ϕ_{Mg2Ca}:

$${ϕ}_{Mg 2 Ca} = {n}_{V , Mg 2 Ca} \frac{4}{3} π {( \frac{{d}_{P}}{2} )}^{3} ,$$

where d_{P} is the mean area equivalent diameter of the Mg₂Ca particles and π is the mathematical constant giving the ratio of a circle's circumference to its diameter.

In a next step, the mass fraction of the Mg₂Ca particles, w_{Mg2Ca} is computed by

$${w}_{Mg 2 Ca} = {ϕ}_{Mg 2 Ca} \frac{{ρ}_{Mg 2 Ca}}{ρ} ,$$

where ρ_{Mg2Ca} is the Mg₂Ca particles' average volumetric mass density and ρ the alloy's average volumetric mass density.

By further using the molar mass of Mg and Ca and the average molar mass of the alloy, the mass fraction of the Mg₂Ca particles can be transformed to the mole fraction of the Mg₂Ca particles as present in the alloy, x_{Mg2Ca}:

$${x}_{Mg 2 Ca} = {w}_{Mg 2 Ca} \frac{\overline{M}}{{M}_{Mg 2 Ca}} ,$$

where M̅ is the alloy's average molar mass and M_{Mg2Ca} the molar mass of the Mg₂Ca intermetallic phase.

It is recommended to choose a field of view for the TEM micrographs to allow for the detection of at least 30 particles of the Mg₂Ca intermetallic phase. From experience, such a field of view typically measures at least 3 micrometer times 3 micrometer. Additionally, the whole process shall be repeated at least three times at samples from different places within the material and the final mole fraction shall be determined by averaging.

The alloy preferably comprises no ternary intermetallic phase, particular no ternary phase comprising magnesium, calcium and zinc. This absence of said ternary phases is considered as being especially beneficial due to the above-described detrimental influence of such ternary phases on corrosion resistance and in vivo hydrogen gas evolution. This is accompanied by a generally preferable absence of the element zinc, since the relatively large difference in the electrochemical potential of zinc compared to the magnesium matrix again can lead to an increased rate of degradation and associated undesirable hydrogen gas evolution upon contact with an aqueous solution, such as body fluids.

If applicable, the alloy preferably comprises 0.5% by weight or less or 0.3% by weight or less or 0.1% by weight or less of zirconium based on the total weight of the alloy. Additionally, or alternatively the alloy preferably comprises between 0.005% by weight and 0.5% by weight zirconium based on the total weight of the alloy, such as between 0.005% by weight and 0.3% by weight zirconium based on the total weight of the alloy or between 0.005% and 0.1% by weight zirconium based on the total weight of the alloy, for instance between 0.01% by weight and 0.1% by weight zirconium based on the total weight of the alloy, more preferably between 0.01% by weight and 0.07% by weight zirconium based on the total weight of the alloy. Additionally, or alternatively, the alloy can comprise 0.5% by weight or less or 0.3% by weight or less or 0.1% by weight or less of hafnium based on the total weight of the alloy. Additionally, or alternatively the alloy preferably comprises between 0.005% by weight and 0.5% by weight hafnium based on the total weight of the alloy, such as between 0.005% by weight and 0.3% by weight hafnium based on the total weight of the alloy or between 0.005% and 0.1% by weight hafnium based on the total weight of the alloy, for instance between 0.01% by weight and 0.1% by weight hafnium based on the total weight of the alloy, more preferably between 0.01% by weight and 0.07% by weight hafnium based on the total weight of the alloy.

Due to the exceptional chemical similarity of hafnium and zirconium, an interchangeability of the two chemical elements can be assumed. The addition of zirconium and/or hafnium leads to a stronger grain-refinement effect during solidification of the alloy from melt compared to Mg—Ca without zirconium and/or hafnium. For minute additions of less than about 0.5% zirconium and/or hafnium by weight based on the total weight of the alloy, this is preferably caused by grain-growth restriction by the then preferably fully dissolved zirconium and/or hafnium. The resulting finer average grain size, in turn, leads to an additional grain-refinement effect during extrusion, additionally to an already increased grain-refinement effect caused by grain-boundary pinning by the presence of Mg₂Ca particles. In summary, a stronger material can be achieved and, important from a technological point of view, the process window to achieve the desired materials properties can be significantly extended by the addition of zirconium and/or hafnium. This is important because of the limited temperature stability of Mg₂Ca intermetallic particles.

In the event that zirconium and/or hafnium is added to the magnesium and calcium, the alloy can comprise zirconium and/or hafnium, preferably being completely dissolved within the magnesium matrix of the alloy. The term “matrix” is well known to the art and refers to the alloy's homogeneous and monolithic microstructure. However, depending on the generation of the billet, finely dispersed intermetallic particles such as zirconium-comprising particles and/or zirconium-comprising clusters and/or hafnium-comprising particles and/or hafnium-comprising clusters and/or zirconium-hafnium-comprising particles and/or zirconium-hafnium-comprising clusters can be present after the generation of the billet and thus in the alloy. These intermetallic particles or clusters may originate from the magnesium-zirconium master alloy and/or magnesium-hafnium master alloy and/or magnesium-zirconium-hafnium master alloy that can be used to introduce zirconium and/or hafnium into the intermediate alloy and consequently also into the final alloy.

That is to say, additionally to the above mentioned Mg₂Ca intermetallic particles, the alloy can comprise intermetallic particles comprising zirconium and/or hafnium.

In any case, it is preferred that a remainder of the alloy consists of magnesium, apart from any impurities, if any. For instance, if the alloy comprises 1% by weight of calcium based on the total weight of the alloy and 0.1% by weight of zirconium based on the total weight of the alloy, the remainder of the alloy would be 98.9% by weight of magnesium based on the total weight of the alloy, possibly including some impurities.

Hence, a preferred Mg—Ca alloy comprises between 99% by weight to 99.85% by weight of Mg and between 0.15% by weight to 1% by weight of Ca, the remainder being impurities, if any, and wherein an amount of the impurities is 0.02% by weight of impurities based on the total weight of the alloy or less, see further below. In other words, a preferred Mg—Ca alloy comprises at least 99% by weight of Mg and at least 0.15% by weight of Ca.

For the sake of simplicity this possible remainder of impurities in the amount of 0.02% by weight of impurities based on the total weight of the alloy or less is not explicitly referred to in the following.

Said Mg—Ca alloy of high strength is preferably produced in the method according to the invention by extruding the billet at least once at an extrusion temperature in the range of 300° C. to 400° C., and at a ram speed in the range of 0.05 mm/s to 0.2 mm/s, and at an extrusion ratio in the range of 50 to 100. Said high strength is associated with an ultimate tensile strength of 300 MPa or more/in the range of 300 MPa to 430 MPa and an elongation at fracture in the range of 2% to 15%.

Said Mg—Ca alloy of high ductility is preferably produced in the method according to the invention by extruding the billet at least once at an extrusion temperature in the range of 360° C. to 430° C., and at a ram speed in the range of 0.15 mm/s to 0.6 mm/s, and at an extrusion ratio in the range of 50 to 100. Said high ductility is associated with an ultimate tensile strength of 150 MPa or more/in the range of 150 MPa to 250 MPa and an elongation at fracture in the range of 20% to 40%.

Said Mg—Ca-alloy having an optimized combination of strength and ductility is preferably produced in the method according to the invention by extruding the billet at least once at an extrusion temperature in the range of 330° C. to 400° C., and at a ram speed in the range of 0.15 mm/s to 0.5 mm/s, and at an extrusion ratio in the range of 50 to 100. Said optimized combination of strength and ductility is associated with an ultimate tensile strength of 250 MPa or more/in the range of 250 MPa to 350 MPa and an elongation at fracture in the range of 8% to 25%.

A more preferred Mg—Ca alloy comprises between 99.35% by weight to 99.75% by weight of Mg and between 0.25% by weight to 0.65% by weight of Ca. In other words, a more preferred Mg—Ca alloy comprises at least 99.35% by weight of Mg and at least 0.25% by weight of Ca.

Said Mg—Ca alloy of high strength is preferably produced in the method according to the invention by extruding the billet at least once at an extrusion temperature in the range of 330° C. to 380° C., and at a ram speed in the range of 0.05 mm/s to 0.15 mm/s, and at an extrusion ratio in the range of 65 to 80. Said high strength is associated with an ultimate tensile strength of 350 MPa or more/in the range of 350 MPa to 430 MPa and an elongation at fracture in the range of 2% to 10%.

Said Mg—Ca alloy of high ductility is preferably produced in the method according to the invention by extruding the billet at least once at an extrusion temperature in the range of 370° C. to 390° C., and at a ram speed in the range of 0.2 mm/s to 0.55 mm/s, and at an extrusion ratio in the range of 65 to 80. Said high ductility is associated with an ultimate tensile strength of 200 MPa or more/in the range of 200 MPa to 250 MPa and an elongation at fracture in the range of 25% to 36%.

Said Mg—Ca alloy having an optimized combination of strength and ductility is preferably produced in the method according to the invention by extruding the billet at least once at an extrusion temperature in the range of 330° C. to 360° C., and at a ram speed in the range of 0.2 mm/s to 0.3 mm/s, and at an extrusion ratio in the range of 65 to 80. Said optimized combination of strength and ductility is associated with an ultimate tensile strength of 270 MPa or more/in the range of 270 MPa to 330 MPa and an elongation at fracture in the range of 10% to 23%.

A particularly preferred Mg—Ca alloy consists of 99.55% by weight of Mg and 0.45% by weight of Ca.

Said Mg—Ca alloy of high strength is preferably produced in the method according to the invention by extruding the billet at least once at an extrusion temperature in the range of 330° C. to 350° C., and at a ram speed in the range of 0.05 mm/s to 0.1 mm/s, and at an extrusion ratio in the range of 65 to 80. Said high strength is associated with an ultimate tensile strength of 380 MPa or more/in the range of 380 MPa to 430 MPa and an elongation at fracture in the range of 3% to 8%.

Said Mg—Ca alloy of high ductility is preferably produced in the method according to the invention by extruding the billet at least once at an extrusion temperature in the range of 380° C. to 390° C., and at a ram speed in the range of 0.3 mm/s to 0.55 mm/s, and at an extrusion ratio in the range of 65 to 80. Said high ductility is associated with an ultimate tensile strength of 200 MPa or more/in the range of 200 MPa to 250 MPa and an elongation at fracture in the range of 30% to 36%.

Said Mg—Ca alloy having an optimized combination of strength and ductility is preferably produced in the method according to the invention by extruding the billet at least once at an extrusion temperature in the range of 340° C. to 360° C., and at a ram speed in the range of 0.25 mm/s to 0.3 mm/s, and at an extrusion ratio in the range of 65 to 80. Said optimized combination of strength and ductility is associated with an ultimate tensile strength of 270 MPa or more/in the range of 270 MPa to 300 MPa and an elongation at fracture in the range of 12% to 18%.

A preferred Mg—Ca—Zr alloy comprises between 98.5% by weight to 99.845% by weight of Mg, between 0.15% by weight to 1% by weight of Ca, and between 0.005% by weight to 0.5% by weight of Zr. In other words, a preferred Mg—Ca—Zr alloy comprises at least 98.5% by weight of Mg, at least 0.15% by weight of Ca, and at least 0.005% by weight of Zr.

Said Mg—Ca—Zr alloy of high strength is preferably produced in the method according to the invention by extruding the billet at least once at an extrusion temperature in the range of 320° C. to 420° C., and at a ram speed in the range of 0.05 mm/s to 0.2 mm/s, and at an extrusion ratio in the range of 50 to 100. Said high strength is associated with an ultimate tensile strength of 300 MPa or more/in the range of 300 MPa to 430 MPa and an elongation at fracture in the range of 2% to 15%.

Said Mg—Ca—Zr alloy of high ductility is preferably produced in the method according to the invention by extruding the billet at least once at an extrusion temperature in the range of 380° C. to 450° C., and at a ram speed in the range of 0.15 mm/s to 0.6 mm/s, and at an extrusion ratio in the range of 50 to 100. Said high ductility is associated with an ultimate tensile strength of 150 MPa or more/in the range of 150 MPa to 250 MPa and an elongation at fracture in the range of 20% to 40%.

Said Mg—Ca—Zr-alloy having an optimized combination of strength and ductility is preferably produced in the method according to the invention by extruding the billet at least once at an extrusion temperature in the range of 350° C. to 420° C., and at a ram speed in the range of 0.15 mm/s to 0.5 mm/s, and at an extrusion ratio in the range of 50 to 100. Said optimized combination of strength and ductility is associated with an ultimate tensile strength of 250 MPa or more/in the range of 250 MPa to 350 MPa and an elongation at fracture in the range of 8% to 25%.

A more preferred Mg—Ca—Zr alloy comprises between 99.25% by weight to 99.745% by weight of Mg, between 0.25% by weight to 0.65% by weight of Ca, and between 0.005% by weight to 0.1% by weight of Zr. In other words, a more preferred Mg—Ca—Zr alloy comprises at least 99.25% by weight of Mg, at least 0.25% by weight of Ca, and at least 0.005% by weight of Zr.

Said Mg—Ca—Zr alloy of high strength is preferably produced in the method according to the invention by extruding the billet at least once at an extrusion temperature in the range of 350° C. to 400° C., and at a ram speed in the range of 0.05 mm/s to 0.15 mm/s, and at an extrusion ratio in the range of 65 to 80. Said high strength is associated with an ultimate tensile strength of 350 MPa or more/in the range of 350 MPa to 430 MPa and an elongation at fracture in the range of 2% to 10%.

Said Mg—Ca—Zr alloy of high ductility is preferably produced in the method according to the invention by extruding the billet at least once at an extrusion temperature in the range of 390° C. to 410° C., and at a ram speed in the range of 0.2 mm/s to 0.55 mm/s, and at an extrusion ratio in the range of 65 to 80. Said high ductility is associated with an ultimate tensile strength of 200 MPa or more/in the range of 200 MPa to 250 MPa and an elongation at fracture in the range of 25% to 36%.

Said Mg—Ca—Zr alloy having an optimized combination of strength and ductility is preferably produced in the method according to the invention by extruding the billet at least once at an extrusion temperature in the range of 350° C. to 380° C., and at a ram speed in the range of 0.2 mm/s to 0.3 mm/s, and at an extrusion ratio in the range of 65 to 80. Said optimized combination of strength and ductility is associated with an ultimate tensile strength of 270 MPa or more/in the range of 270 MPa to 330 MPa and an elongation at fracture in the range of 10% to 23%.

A particularly preferred Mg—Ca—Zr alloy consists of 99.48% by weight of Mg, 0.45% by weight of Ca, and 0.07% by weight of Zr.

Said Mg—Ca—Zr alloy of high strength is preferably produced in the method according to the invention by extruding the billet at least once at an extrusion temperature in the range of 350° C. to 370° C., and at a ram speed in the range of 0.05 mm/s to 0.1 mm/s, and at an extrusion ratio in the range of 65 to 80. Said high strength is associated with an ultimate tensile strength of 380 MPa or more/in the range of 380 MPa to 430 MPa and an elongation at fracture in the range of 3% to 8%.

Said Mg—Ca—Zr alloy of high ductility is preferably produced in the method according to the invention by extruding the billet at least once at an extrusion temperature in the range of 400° C. to 410° C., and at a ram speed in the range of 0.3 mm/s to 0.55 mm/s, and at an extrusion ratio in the range of 65 to 80. Said high ductility is associated with an ultimate tensile strength of 200 MPa or more/in the range of 200 MPa to 250 MPa and an elongation at fracture in the range of 30% to 36%.

Said Mg—Ca—Zr alloy having an optimized combination of strength and ductility is preferably produced in the method according to the invention by extruding the billet at least once at an extrusion temperature in the range of 360° C. to 380° C., and at a ram speed in the range of 0.25 mm/s to 0.3 mm/s, and at an extrusion ratio in the range of 65 to 80. Said optimized combination of strength and ductility is associated with an ultimate tensile strength of 270 MPa or more/in the range of 270 MPa to 300 MPa and an elongation at fracture in the range of 12% to 18%.

A preferred Mg—Ca—Hf alloy comprises between 98.5% by weight to 99.845% by weight of Mg, between 0.15% by weight to 1% by weight of Ca, and between 0.005% by weight to 0.5% by weight of Hf. In other words, a preferred Mg—Ca—Hf alloy comprises at least 98.5% by weight of Mg, at least 0.15% by weight of Ca, and at least 0.005% by weight of Hf.

Said Mg—Ca—Hf alloy of high strength is preferably produced in the method according to the invention by extruding the billet at least once at an extrusion temperature in the range of 320° C. to 420° C., and at a ram speed in the range of 0.05 mm/s to 0.2 mm/s, and at an extrusion ratio in the range of 50 to 100. Said high strength is associated with an ultimate tensile strength of 300 MPa or more/in the range of 300 MPa to 430 MPa and an elongation at fracture in the range of 2% to 15%.

Said Mg—Ca—Hf alloy of high ductility is preferably produced in the method according to the invention by extruding the billet at least once at an extrusion temperature in the range of 380° C. to 450° C., and at a ram speed in the range of 0.15 mm/s to 0.6 mm/s, and at an extrusion ratio in the range of 50 to 100. Said high ductility is associated with an ultimate tensile strength of 150 MPa or more/in the range of 150 MPa to 250 MPa and an elongation at fracture in the range of 20% to 40%.

Said Mg—Ca—Hf-alloy having an optimized combination of strength and ductility is preferably produced in the method according to the invention by extruding the billet at least once at an extrusion temperature in the range of 350° C. to 420° C., and at a ram speed in the range of 0.15 mm/s to 0.5 mm/s, and at an extrusion ratio in the range of 50 to 100. Said optimized combination of strength and ductility is associated with an ultimate tensile strength of 250 MPa or more/in the range of 250 MPa to 350 MPa and an elongation at fracture in the range of 8% to 25%.

A more preferred Mg—Ca—Hf alloy comprises between 99.25% by weight to 99.745% by weight of Mg, between 0.25% by weight to 0.65% by weight of Ca, and between 0.005% by weight to 0.1% by weight of Hf. In other words, a more preferred Mg—Ca—Hf alloy comprises at least 99.25% by weight of Mg, at least 0.25% by weight of Ca, and at least 0.005% by weight of Hf.

Said Mg—Ca—Hf alloy of high strength is preferably produced in the method according to the invention by extruding the billet at least once at an extrusion temperature in the range of 350° C. to 400° C., and at a ram speed in the range of 0.05 mm/s to 0.15 mm/s, and at an extrusion ratio in the range of 65 to 80. Said high strength is associated with an ultimate tensile strength of 350 MPa or more/in the range of 350 MPa to 430 MPa and an elongation at fracture in the range of 2% to 10%.

Said Mg—Ca—Hf alloy of high ductility is preferably produced in the method according to the invention by extruding the billet at least once at an extrusion temperature in the range of 390° C. to 410° C., and at a ram speed in the range of 0.2 mm/s to 0.55 mm/s, and at an extrusion ratio in the range of 65 to 80. Said high ductility is associated with an ultimate tensile strength of 200 MPa or more/in the range of 200 MPa to 250 MPa and an elongation at fracture in the range of 25% to 36%.

Said Mg—Ca—Hf alloy having an optimized combination of strength and ductility is preferably produced in the method according to the invention by extruding the billet at least once at an extrusion temperature in the range of 350° C. to 380° C., and at a ram speed in the range of 0.2 mm/s to 0.3 mm/s, and at an extrusion ratio in the range of 65 to 80. Said optimized combination of strength and ductility is associated with an ultimate tensile strength of 270 MPa or more/in the range of 270 MPa to 330 MPa and an elongation at fracture in the range of 10% to 23%.

A particularly preferred Mg—Ca—Hf alloy consists of 99.48% by weight of Mg, 0.45% by weight of Ca, and 0.07% by weight of Hf.

Said Mg—Ca—Hf alloy of high strength is preferably produced in the method according to the invention by extruding the billet at least once at an extrusion temperature in the range of 350° C. to 370° C., and at a ram speed in the range of 0.05 mm/s to 0.1 mm/s, and at an extrusion ratio in the range of 65 to 80. Said high strength is associated with an ultimate tensile strength of 380 MPa or more/in the range of 380 MPa to 430 MPa and an elongation at fracture in the range of 3% to 8%.

Said Mg—Ca—Hf alloy of high ductility is preferably produced in the method according to the invention by extruding the billet at least once at an extrusion temperature in the range of 400° C. to 410° C., and at a ram speed in the range of 0.3 mm/s to 0.55 mm/s, and at an extrusion ratio in the range of 65 to 80. Said high ductility is associated with an ultimate tensile strength of 200 MPa or more/in the range of 200 MPa to 250 MPa and an elongation at fracture in the range of 30% to 36%.

Said Mg—Ca—Hf alloy having an optimized combination of strength and ductility is preferably produced in the method according to the invention by extruding the billet at least once at an extrusion temperature in the range of 360° C. to 380° C., and at a ram speed in the range of 0.25 mm/s to 0.3 mm/s, and at an extrusion ratio in the range of 65 to 80. Said optimized combination of strength and ductility is associated with an ultimate tensile strength of 270 MPa or more/in the range of 270 MPa to 300 MPa and an elongation at fracture in the range of 12% to 18%.

A preferred Mg—Ca—Zr—Hf alloy comprises between 98.5% by weight to 99.845% by weight of Mg, between 0.15% by weight to 1% by weight of Ca, and in total between 0.005% by weight to 0.5% by weight of Zr and Hf. In other words, a preferred Mg—Ca—Zr—Hf alloy comprises at least 98.5% by weight of Mg, at least 0.15% by weight of Ca, and in total at least 0.005% by weight of Zr and Hf.

Said Mg—Ca—Zr—Hf alloy of high strength is preferably produced in the method according to the invention by extruding the billet at least once at an extrusion temperature in the range of 320° C. to 420° C., and at a ram speed in the range of 0.05 mm/s to 0.2 mm/s, and at an extrusion ratio in the range of 50 to 100. Said high strength is associated with an ultimate tensile strength of 300 MPa or more/in the range of 300 MPa to 430 MPa and an elongation at fracture in the range of 2% to 15%.

Said Mg—Ca—Zr—Hf alloy of high ductility is preferably produced in the method according to the invention by extruding the billet at least once at an extrusion temperature in the range of 380° C. to 450° C., and at a ram speed in the range of 0.15 mm/s to 0.6 mm/s, and at an extrusion ratio in the range of 50 to 100. Said high ductility is associated with an ultimate tensile strength of 150 MPa or more/in the range of 150 MPa to 250 MPa and an elongation at fracture in the range of 20% to 40%.

Said Mg—Ca—Zr—Hf-alloy having an optimized combination of strength and ductility is preferably produced in the method according to the invention by extruding the billet at least once at an extrusion temperature in the range of 350° C. to 420° C., and at a ram speed in the range of 0.15 mm/s to 0.5 mm/s, and at an extrusion ratio in the range of 50 to 100. Said optimized combination of strength and ductility is associated with an ultimate tensile strength of 250 MPa or more/in the range of 250 MPa to 350 MPa and an elongation at fracture in the range of 8% to 25%.

A more preferred Mg—Ca—Zr—Hf alloy comprises between 99.25% by weight to 99.74% by weight of Mg, between 0.25% by weight to 0.65% by weight of Ca, and in total between 0.01% by weight to 0.1% by weight of Zr and Hf. In other words, a more preferred Mg—Ca—Zr—Hf alloy comprises at least 99.25% by weight of Mg, at least 0.25% by weight of Ca, at least 0.005% by weight of Zr, and at least 0.005% by weight of Hf.

Said Mg—Ca—Zr—Hf alloy of high strength is preferably produced in the method according to the invention by extruding the billet at least once at an extrusion temperature in the range of 350° C. to 400° C., and at a ram speed in the range of 0.05 mm/s to 0.15 mm/s, and at an extrusion ratio in the range of 65 to 80. Said high strength is associated with an ultimate tensile strength of 350 MPa or more/in the range of 350 MPa to 430 MPa and an elongation at fracture in the range of 2% to 10%.

Said Mg—Ca—Zr—Hf alloy of high ductility is preferably produced in the method according to the invention by extruding the billet at least once at an extrusion temperature in the range of 390° C. to 410° C., and at a ram speed in the range of 0.2 mm/s to 0.55 mm/s, and at an extrusion ratio in the range of 65 to 80. Said high ductility is associated with an ultimate tensile strength of 200 MPa or more/in the range of 200 MPa to 250 MPa and an elongation at fracture in the range of 25% to 36%.

Said Mg—Ca—Zr—Hf alloy having an optimized combination of strength and ductility is preferably produced in the method according to the invention by extruding the billet at least once at an extrusion temperature in the range of 350° C. to 380° C., and at a ram speed in the range of 0.2 mm/s to 0.3 mm/s, and at an extrusion ratio in the range of 65 to 80. Said optimized combination of strength and ductility is associated with an ultimate tensile strength of 270 MPa or more/in the range of 270 MPa to 330 MPa and an elongation at fracture in the range of 10% to 23%.

A particularly preferred Mg—Ca—Zr—Hf alloy consists of 99.48% by weight of Mg, 0.45% by weight of Ca, and in total 0.07% by weight of Zr and Hf.

Said Mg—Ca—Zr—Hf alloy of high strength is preferably produced in the method according to the invention by extruding the billet at least once at an extrusion temperature in the range of 350° C. to 370° C., and at a ram speed in the range of 0.05 mm/s to 0.1 mm/s, and at an extrusion ratio in the range of 65 to 80. Said high strength is associated with an ultimate tensile strength of 380 MPa or more/in the range of 380 MPa to 430 MPa and an elongation at fracture in the range of 3% to 8%.

Said Mg—Ca—Zr—Hf alloy of high ductility is preferably produced in the method according to the invention by extruding the billet at least once at an extrusion temperature in the range of 400° C. to 410° C., and at a ram speed in the range of 0.3 mm/s to 0.55 mm/s, and at an extrusion ratio in the range of 65 to 80. Said high ductility is associated with an ultimate tensile strength of 200 MPa or more/in the range of 200 MPa to 250 MPa and an elongation at fracture in the range of 30% to 36%.

Said Mg—Ca—Zr—Hf alloy having an optimized combination of strength and ductility is preferably produced in the method according to the invention by extruding the billet at least once at an extrusion temperature in the range of 360° C. to 380° C., and at a ram speed in the range of 0.25 mm/s to 0.3 mm/s, and at an extrusion ratio in the range of 65 to 80. Said optimized combination of strength and ductility is associated with an ultimate tensile strength of 270 MPa or more/in the range of 270 MPa to 300 MPa and an elongation at fracture in the range of 12% to 18%.

The method can furthermore comprise the step of purifying prior to the generation of the billet. That is, the magnesium and/or the calcium can be purified preferably by vacuum distillation prior to the generation of the billet.

In this regard it should be noted that purification of the alloy can be to a level such that an amount of impurities is 0.02% by weight of impurities based on the total weight of the alloy or less, for instance 0.01% by weight of impurities based on the total weight of the alloy or less or even 0.002% by weight of impurities based on the total weight of the alloy or less. The purification required for specific applications depends on the particular trace element. For instance, individual purifications to levels being below 0.002% by weight or even below 0.0002% by weight of a particular impurity based on the total weight of the alloy were obtained for the trace elements copper, iron, nickel and cobalt. A purification to the level being about 0.01% by weight of zinc based on the total weight of the alloy or less was obtained for the trace element zinc. In fact, even lower amounts of zinc impurities were achieved, for instance an amount of zinc being 0.0005% or less by weight of zinc based on the total weight of the alloy.

It is furthermore conceivable that the method comprises the step of generating the billet by vacuum distillation.

In this regard it should be noted that the method can comprise the combined step of purifying and generating the billet by vacuum distillation. In other words, the method can comprise the step of simultaneous distillation and alloying. Said simultaneous step of distillation and alloying preferably corresponds to the step of simultaneous distillation and alloying as described in the PCT application PCT/EP 2021/053337, which is herein incorporated by reference. That is to say, the magnesium and calcium can be received in a trough or the like being provided in a chamber, whereupon the chamber is evacuated and heated such that the magnesium and calcium being received in the trough are vaporized so as to form vapor. Said vapor is thereafter preferably condensed to form a condensate, and which condensate in turn is preferably received in a collecting vessel and allowed to solidify, whereby the billet is formed. That is to say, it is conceivable to purify and mix an alloy comprising magnesium and calcium by the same process based on distillation, whereby the billet is generated.

However, it is likewise conceivable to generate the billet by other processes, such as in a conventional melting and/or casting process. It should be noted that in this case it is however nevertheless conceivable that the method comprises the step of purifying the magnesium and/or calcium prior to the generation of the billet as described above.

Furthermore, in any case, it is conceivable that the billet is generated within a chamber that is filled with an inert gas, preferably with argon. That is, either the billet generation by means of the vacuum distillation or by means of other processes such as the melting process can be performed within a chamber that is filled with an inert gas. A pressure of the inert gas preferably is in the range of 300 mbar to 800 mbar. However, other pressures are likewise conceivable, for instance a pressure of 10 mbar or more.

As just mentioned, the billet can be generated by means of a melting process. Hence, the method can comprise the step of generating the billet by melting calcium and magnesium and preferably additionally zirconium and/or hafnium, whereby a melt comprising calcium and magnesium and preferably additionally zirconium and/or hafnium is formed, and by subsequently solidifying said melt.

To this end it is preferred that zirconium is provided in the form of a magnesium-zirconium master alloy, i.e. a Mg—Zr master alloy, and/or that hafnium is preferably provided in the form of a magnesium-hafnium master alloy, i.e. a Mg—Hf master alloy.

For instance, a hole could be made into the magnesium and calcium could be added into said hole. If applicable, also the Mg—Zr master alloy and/or the Mg—Hf master alloy could be added into said hole. These materials are preferably added into the magnesium in its initial state, i.e. into the magnesium raw material, or, if applicable, into the magnesium after its purification. It is furthermore preferred that these materials are added to said hole before a melting temperature of the magnesium is reached. The addition of these materials to the magnesium is preferably done at room temperature. However, it is likewise conceivable that the addition occurs at another or elevated temperature.

The melt is preferably generated by heating the materials to a melting temperature, wherein said melting temperature is preferably in the range of 650° C. to 900° C., more preferably in the range of 650° C. to 750° C. The melt can be formed by inductive heating, although other heating methods known in the art are likewise conceivable. It is furthermore preferred to maintain the melt at the melting temperature or above for a time period in the range of 1 minute to 100 minutes, although longer times are conceivable. Furthermore, the melt can be stirred, for instance by currents being induced by inductive heating and/or by mechanical stirring and/or by the irradiation of ultrasonic waves.

The melt is preferably solidified by generating the melt in a crucible and by arranging said crucible subsequently on a cooling element. The cooling element preferably is a block of material such as copper or a copper alloy, the cooling element preferably being actively cooled. However, it is likewise conceivable that the solidification of the melt is achieved by casting.

The cooling element preferably has a high thermal conductivity such as a thermal conductivity being at least 150 W/m K and/or a high specific heat capacity such as a specific heat capacity being at least 24 J/mol K. The cooling element preferably furthermore is at a temperature being lower than the temperature of the crucible at the time of arranging the crucible on the cooling element. A difference in temperature between the cooling element and the crucible at the time of arranging the crucible on the cooling element preferably is 100° C. or more. The cooling element can be actively cooled, for example by supplying a cooling fluid such as water, preferably at room temperature, to the cooling element.

The method can further comprise at least one step of homogenization annealing heat treatment being performed after the billet is generated and before the billet is extruded. To this end it is particularly preferred that the calcium and preferably additionally also the zirconium and/or the hafnium are brought into solid solution. An annealing temperature preferably is in the range of 350° C. to 520° C. A holding period during which the annealing temperature is maintained preferably is 0.5 hours or more, for instance between 0.5 hours and 50 hours, although other holding periods are likewise conceivable. Moreover, it is conceivable that two or more annealing steps are performed. In addition, when two or more annealing steps are performed, an annealing temperature of these steps can differ. In fact, it is preferred to perform two or more annealing steps at successively increasing annealing temperatures. Preferred temperature differences between two consecutive annealing steps are 100° C. or more.

The method can further comprise the step of quenching the billet after a last step of annealing. The step of quenching preferably comprises the supplying of a pressurized gas or a liquid to the billet. A preferred liquid is water.

The billet is preferably preheated prior to the extrusion. The billet is particularly preferably preheated to the extrusion temperature at which the extrusion will be performed.

It is furthermore preferred that Mg₂Ca intermetallic particles are formed prior to the extrusion and preferably during the preheating of the billet. In this regard it should be noted that Mg₂Ca intermetallic particles can form during solidification. However, according to the Mg—Ca phase diagram Mg₂Ca will be dissolved during the heat treatments before extrusion if heated to 520° C. or more, for instance. But, at higher Ca contents and if lower annealing temperatures are chosen, Mg₂Ca intermetallic particles might remain after homogenization. In this case, these intermetallic particles can grow and additional precipitates can form during the preheating.

After the heat treatments and before the extrusion of the billet a super-saturated solid solution of calcium in magnesium can be present due to quenching. The billet is preferably preheated prior to the extrusion. The billet is particularly preferably preheated to the extrusion temperature at which the extrusion will be performed. During this preheating, Mg₂Ca intermetallic particles can precipitate, beginning with small clusters that get bigger in size, but smaller in number. However, it is likewise conceivable that the precipitation of Mg₂Ca intermetallic particles is achieved during a separate precipitation heat treatment after homogenization heat treatments and before the preheating prior to the extrusion. This precipitation heat treatment is performed at a temperature below the solvus temperature of the Mg₂Ca intermetallic particles, which depends on the calcium content of the alloy. The longer this precipitation heat treatment or the preheating prior to extrusion lasts, the larger the precipitates can get and the less they will be. The same holds true for higher temperatures as long as they will be below the solvus temperature of the Mg₂Ca intermetallic particles. Especially when an alloy of high strength is desired it is preferred to select the method parameters such, that the Mg₂Ca intermetallic particles are as small and as much as possible because the Zener grain-pinning effect (that is responsible for the strength increase during extrusion) gets stronger with small particles and large numbers. In addition, the size and the number density of the Mg₂Ca intermetallic particles can be affected by the extrusion parameters. In particular, by preheating the billet to a higher extrusion temperature the size and number density of the Mg₂Ca intermetallic particles will be different as compared to lower extrusion temperatures.

The Mg₂Ca intermetallic particles preferably have a size of 500 nanometers or less, preferably of 200 nanometers or less, particularly preferably of 100 nanometers or less. As mentioned earlier, the size of the Mg₂Ca intermetallic particles is preferably determined using image analysis software preferably according to ISO 13322-1.

To this end it is preferred that not only the billet but also the alloy, i.e. the final alloy or target alloy, comprises these Mg₂Ca intermetallic particles.

In this regard it should be noted that Mg₂Ca intermetallic particles can also form during the alloying step at solidification from the melt or during the cooling period after solidification. However, according to the Mg—Ca phase diagram Mg₂Ca will be dissolved during the homogenization heat treatments before extrusion if temperatures above the solvus temperature of the Mg₂Ca intermetallic particles are chosen. At higher Ca contents and if lower annealing temperatures are chosen, Mg₂Ca intermetallic particles might remain after homogenization heat treatment. In this case, these intermetallic particles can grow, and additional precipitates can form during the precipitation heat treatment or preheating prior to extrusion.

The alloy preferably forms a fine-grained structure with an average grain size of 5 micrometers or less, preferably of 3 micrometers or less such as of 1 micrometer or less, for instance of 500 nanometers or less. The average grain size is determined from metallographic images that can be produced by optical microscopy or electron microscopy including techniques such as Electron Backscatter Diffraction or Transmission Kikuchi Diffraction, and according to the Lineal Intercept Method as described in ASTM E112—Standard Test Methods for Determining Average Grain Size. The term “average grain size” as used here is to be understood as to be the “mean lineal intercept length” as defined in ASTM E112. In fact, it is preferred that the alloy for high-strength applications, e.g. an alloy having an ultimate tensile strength of 300 MPa or more and an elongation at fracture in the range of 2% to 15%, or an alloy having an ultimate tensile strength of 350 MPa or more and an elongation at fracture in the range of 2% to 10%, has a fine-grained structure with an average grain size of 1 micrometer or less, for instance of 500 nanometers or less. An alloy having a high ductility, for instance an alloy having an ultimate tensile strength of 150 MPa or more and an elongation at fracture of 20% or more, preferably has a larger fine-grained structure, for instance a fine-grained structure with an average grain size of 5 micrometers or less, preferably of 3 micrometers or less. An alloy having an optimized combination of strength and ductility, for instance having an ultimate tensile strength in the range of 250 MPa to 350 MPa and an elongation at fracture in the range of 8% to 25%, preferably has an intermediate fine-grained structure, for instance a fine-grained structure with an average grain size of 3 micrometers or less, preferably of 2 micrometers or less, for instance 1 micrometer or less.

Since the ultimate tensile strength and the elongation at fracture can be determined by the particular extrusion parameters, the said average grain size of 5 micrometers or less, preferably of 3 micrometers or less such as of 1 micrometer or less, for instance of 500 nanometers or less is obtained by performing the extrusion at the extrusion temperature in the range of 250° C. to 450° C. and at the ram speed in the range of 0.05 mm/s to 1 mm/s and at the extrusion ratio in the range of 20 to 150 and particularly preferably at the extrusion ratio in the range of 35 to 150. In essence, a low extrusion temperature at constant ram speed typically correlates with smaller grain size because of less thermal energy available for grain growth during and after extrusion, resulting in an alloy of higher strength because of the well-known Hall-Petch relationship (see further details below). In addition, at constant temperature, the ram speed can have a severe effect on grain size. With higher ram speed, average grain size can quickly increase. Hence, by the particular selection of these extrusion parameters a desired average grain size can be obtained.

It is furthermore preferred that the Mg₂Ca intermetallic particles are preferably distributed dispersely at grain boundaries of the fine-grained structure and/or in the grains of the fine-grained structure.

If zirconium and/or hafnium is present and in the form of intermetallic particles or clusters, it is furthermore preferred that the finely dispersed Zr-comprising and/or Hf-comprising intermetallic particles are distributed dispersely at grain boundaries of the fine-grained structure and/or in the grains of the fine-grained structure as well.

In the event that intermetallic particles are present, during the step of extrusion, where a severe plastic deformation typically goes along with this step, wherein the intermetallic particles present in the alloy provide resistance against grain growth—being known as Zener pinning—and thereby keeping grains small.

The Zener pinning pressure is given as

$${P}_{s} = \frac{3 {F}_{v} γ}{2 r}$$

with Pₛ the Zener pinning pressure, Fᵥ the volume fraction of the particles under consideration, γ the grain-boundary energy per unit area and r the average radius of the particles under consideration.

The larger the Zener pinning pressure the stronger is the resistance against moving a grain boundary and consequently restriction of grain growth. So, a higher volume fraction of the pinning particles and/or a smaller size of the pinning particles are favorable for achieving small grain sizes.

Small grains are related to high strength—according to the so-called Hall-Petch relation:

$${σ}_{y} = {σ}_{0} + \frac{{k}_{y}}{\sqrt{d}}$$

where σ_{y} is the yield stress, σ₀ is a materials constant for the starting stress for dislocation movement, k_{y} is the so-called Hall-Petch coefficient and a constant specific to each material, and d is the average grain diameter.

It should be noted that as a hexagonal close-packed material the Hall-Petch coefficient for Mg is exceptionally high, namely about 220 MPa*μm\hat{ }½ exemplarily for Mg—Zn—Ca, therefore grain refining can lead to an especially large increase in strength in case of magnesium alloys.

It should furthermore be noted that not all intermetallic particles that can ultimately be found in the alloy, i.e. in the final alloy or target alloy, must have been created during the pre-heating. Instead, there might be Mg₂Ca intermetallic particles that have been created during solidification of the alloy, or Zr-comprising or Hf-comprising or Zr—Hf-comprising intermetallic particles be present originating from the Mg—Zr and/or Mg—Hf master alloy, or further precipitation (and/or modification of existing intermetallic particles) during the step of extrusion, or further precipitation (and/or modification of existing intermetallic particles) after the step of extrusion.

The method preferably furthermore comprises a step of grain refinement, where grain refinement preferably occurs during the step of extrusion and is preferably produced by the one or more intermetallic particles such as the Mg₂Ca intermetallic particles and/or zirconium-comprising clusters and/or zirconium-comprising particles and/or hafnium-comprising clusters and/or hafnium-comprising particles. In fact, the grain refinement preferably occurs during the step of extrusion by a plastic deformation, subsequent recrystallization, and prevented grain growth by Zener pining due to the present intermetallic particles and potentially Zr and/or Hf in solid solution.

The method preferably further comprises the step of performing a heat treatment of the alloy. The heat treatment is preferably performed after the step of extrusion. The heat treatment is preferably performed at a temperature in the range of 150° C. to 330° C. Additionally or alternatively the heat treatment is preferably performed with a holding period of 30 seconds or more, for instance during a holding period in the range of 30 seconds to 100 hours, although other holding periods are likewise conceivable.

The method can further comprise the step of coating at least part of the alloy, in particular at least part of a surface of the alloy. The coating preferably is a plasma electrolytic oxidation coating and/or a plasma electrolytic anodization coating and/or an amorphous metallic coating and/or a fluoric conversion coating and/or a Mg(OH)₂ coating and/or a calcium phosphate conversion coating and/or a hydroxy-apatite coating and/or an organic coating and/or a biodegradable polymer coating and/or a sol-gel coating. The plasma electrolytic anodization coating or the plasma electrolytic oxidation coating is preferably produced by employing a phosphate-based electrolyte and particularly preferable the electrolyte comprises urea and/or boric acid and/or KOH. The plasma electrolytic oxidation coating process is preferably performed with direct current. Examples of a biodegradable polymer coating are PLA, PLGA, PLLA, PCL or PHB coatings. Likewise, a combination of these coatings is conceivable. Way of applying these coatings are well-known to the person skilled in the art. A thickness of the coating is preferably in the range of 1 micrometer to 20 micrometers, more preferably in the range of 3 micrometers to 12 micrometers. The step of coating is preferably performed after the creation of the final alloy, or target alloy, and/or a heat treatment of the alloy. As mentioned earlier, the alloy is preferably used in or as an implantable medical device such as an implant. In this regard it is particularly preferred that the coating is applied after the final shape of the implant has been created. The final shape of the implant in turn is preferably created by subtractive machining methods, such as milling or turning or grinding and/or potential additional forming methods, such as die forming or drawing.

For illustrative purposes only the conceivable steps of the method according to the invention are summarized in the following. That is, the method of producing the alloy comprising magnesium and calcium plus possibly zirconium and/or hafnium according to the invention can comprise the following steps, that are preferably performed in the given order:
1. Optional step of purification the magnesium and/or the calcium;
2. Step of generating the billet, for instance by vacuum distillation or by melting and by subsequent solidification;
3. Optional Step of annealing also called step of homogenization;
4. Optional Step of quenching;
5. Optional Step of pre-heating;
6. Step of extrusion;
7. Optional Step of heat treatment; and
8. Optional Step of coating.

In another aspect an alloy comprising magnesium and calcium, preferably an alloy as produced as described above, is provided. The alloy has an ultimate tensile strength in the range of 100 MPa to 500 MPa and an elongation at fracture in the range of 2% to 50%, more preferably an ultimate tensile strength in the range of 150 MPa to 500 MPa and an elongation at fracture of 2% to 40%.

Any explanations provided herein with regard to the method of producing the alloy likewise apply to the alloy and vice versa.

For instance, the alloy preferably comprises between 0.15% by weight and 1.0% by weight of calcium based on the total weight of the alloy. Additionally or alternatively, the alloy preferably comprises an intermetallic phase of Mg₂Ca. It is furthermore preferred that the intermetallic phase of Mg₂Ca is in a mole fraction in the range of 0% to 1.5%. That is, the alloy preferably comprises Mg₂Ca intermetallic particles, the Mg₂Ca intermetallic particles preferably having a size of 500 nanometers or less, preferably of 200 nanometers or less, particularly preferably of 100 nanometers or less. It is furthermore preferred that the alloy forms a fine-grained structure with an average grain size of 5 micrometers or less, preferably of 3 micrometers or less such as 1 micrometer or less such as 500 nanometers or less. The Mg₂Ca intermetallic particles are preferably distributed dispersely at grain boundaries of the fine-grained structure and/or in the grains of the fine-grained structure. The alloy can furthermore comprise zirconium and/or hafnium. If applicable, the alloy preferably comprises 0.5% by weight or less or 0.3% by weight or less or 0.1% or less of zirconium based on the total weight of the alloy. Additionally or alternatively the alloy preferably comprises between 0.005% by weight and 0.5% by weight zirconium based on the total weight of the alloy, such as between 0.005% by weight and 0.3% by weight zirconium based on the total weight of the alloy or between 0.005% and 0.1% by weight zirconium based on the total weight of the alloy, for instance between 0.01% by weight and 0.1% by weight zirconium based on the total weight of the alloy, more preferably between 0.01% by weight and 0.07% by weight zirconium based on the total weight of the alloy. Additionally, or alternatively, the alloy can comprise 0.5% by weight or less or 0.3% by weight or less or 0.1% by weight or less of hafnium based on the total weight of the alloy. Additionally or alternatively the alloy preferably comprises between 0.005% by weight and 0.5% by weight hafnium based on the total weight of the alloy, such as between 0.005% by weight and 0.3% by weight hafnium based on the total weight of the alloy or between 0.005% and 0.1% by weight hafnium based on the total weight of the alloy, for instance between 0.01% by weight and 0.1% by weight hafnium based on the total weight of the alloy, more preferably between 0.01% by weight and 0.07% by weight hafnium based on the total weight of the alloy. In the event that zirconium and/or hafnium are present, the alloy can furthermore comprise intermetallic particles of zirconium and/or hafnium, such as finely dispersed zirconium-comprising particles, and/or zirconium-comprising clusters and/or hafnium-comprising particles and/or hafnium-comprising clusters and/or zirconium-hafnium-comprising particles and/or zirconium-hafnium-comprising clusters. In any case, it is preferred that a remainder of the alloy consists of magnesium, apart from any impurities, if any.

The alloy preferably further comprises at least partially a coating, the coating preferably having a thickness in the range of 1 micrometer and 20 micrometers, more preferably in the range of 3 micrometers to 12 micrometers and/or being a plasma electrolytic anodization coating and/or an amorphous metallic coating and/or a fluoric conversion coating and/or a Mg(OH)₂ coating and/or a calcium phosphate conversion coating and/or a hydroxy-apatite coating and/or an organic coating and/or a biodegradable polymer coating and/or a sol-gel coating or a combination those as mentioned above.

The alloy preferably has a degradation rate being smaller than 1 millimeter per year, more preferably being smaller than 0.5 millimeter per year, and particularly preferably being smaller than 0.4 millimeter per year when tested according to the testing standard ASTM F3268. That is, the alloy according to the present invention exhibits these degradation rates when measured from implantation into the bone tissue of an animal for a period of 4 to 8 weeks and by employing mass loss measurements after removal of corrosion products. This low degradation rate can be attributed at least partly to the lack of zinc in the alloy.

In a further aspect the alloy as described above is used as an implantable medical device. The implantable medical device preferably is an implant and/or biodegradable and/or configured for use in orthopedic surgery and/or in dental applications and/or in vascular intervention. Said alloy is preferably obtained in the method as described above. Any explanations made with regard to the method or the alloy likewise apply to the use of the alloy as an implantable medical device and vice versa.

In a further aspect an implantable medical device comprising or consisting of an alloy as described above is provided. The implantable medical device is preferably obtained in the method as described above. The implantable medical device preferably is an implant and/or is biodegradable and/or is configured for use in orthopedic surgery and/or in dental applications and/or in vascular intervention. Any explanations made with regard to the method or the alloy or the use of the alloy likewise apply to the implantable medical device and vice versa.

The alloy or implantable medical device according to the invention is especially suitable for orthopedic and vascular intervention implants where extraordinary biocompatibility is required, for instance for children and adolescence persons, and lowest degradation rate and high demand for ductility is desired. Particularly preferably the alloy or implantable medical device according to the invention is used as or corresponds to plates—that need strong interoperative forming—surgical clips, stents or membranes for guided bone regeneration in dentistry, and porous implants. Likewise preferred are alloys or implantable medical devices according to the invention being used as or corresponding to vascular scaffolds (stent), esophageal stents, urethra stents, surgical clips, bone-tissue regeneration supports, osteosynthesis implants (such as screw, plate, nail, pin, wire), porous implants, membrane for guided bone regenerations, orthopedic implants, drug-delivery containers, osteotomy implants, or veterinary orthopedic implants.

### BRIEF DESCRIPTION OF THE DRAWINGS

Preferred embodiments of the invention are described in the following with reference to the drawings, which are for the purpose of illustrating the present preferred embodiments of the invention and not for the purpose of limiting the same. In the drawings,

FIG. 1 shows a diagram depicting the ultimate tensile strength versus elongation at fracture and ram speed of Mg—Ca alloys according to the invention;

FIG. 2 shows a diagram depicting the stress-strain curves of deliberately chosen alloys according to the invention;

FIG. 3 shows a diagram depicting the elongation at fracture versus ram speed for various extruded lean Mg—Ca alloys according to the invention, demonstrating that over a wide range of Ca-content high elongation at fracture is achievable;

FIG. 4 shows a diagram depicting the ultimate tensile strength versus ram speed for various extruded lean Mg—Ca alloys according to the invention;

FIG. 5 shows electron backscatter diffraction analysis and pole figure exhibiting low grain size and microstructural texture in an alloy according to the invention;

FIG. 6 shows a transmission electron microscopy image exhibiting ultrafine grain structure and nanometer-sized Mg₂Ca intermetallic particles in an alloy according to the invention;

FIG. 7 shows a micro-computed tomography section of an implant according to the invention after 8 weeks in sheep;

FIG. 8 shows a plate and screws according to the invention after 8 weeks of implantation in sheep and chemical removal of degradation products;

FIG. 9 shows a diagram depicting stress-strain curves of extruded Mg—Ca and Mg—Zn—Ca—Zr alloys according to the invention;

FIG. 10 shows microstructure of an extruded Mg—Zn—Ca—Zr alloy according to the invention that has been extruded at an extrusion temperature of 375° C. and at a ram speed of 0.2 mm/s.

FIG. 11 shows the simulated mole fraction of the Mg₂Ca phase in thermodynamic equilibrium in Mg—Ca alloys according to the invention with respect to the Ca-content of the alloy in weight percent based on the total weight of the alloy and temperature.

### DESCRIPTION OF PREFERRED EMBODIMENTS

With respect to the figures, different aspects of the alloys according to the invention will be described in greater detail.

### Lean Binary Mg—Ca Alloys

A total of 13 different billets were successfully melted, divided into three parts and extruded, resulting in 36 different alloys according to the present invention. Additionally, three alloys were prepared with parameters deviating from the ones considered as being optimal, namely an elevated ram speed of 8 mm/s. Compositions and suitable extrusion parameters were chosen based on thermodynamic simulations on the mole fraction of the Mg₂Ca phase as being present in thermodynamic equilibrium (FIG. 11).

The following method of preparation was used:

First, a high-purity (99.5% pure) magnesium ingot was cut into smaller pieces with the aid of a band saw, and these pieces were machined subsequently to fit into a graphite crucible. Additionally, a hole was drilled into the top of the pieces to subsequently accommodate the calcium raw material pieces, with purity of 99% or higher. The assembly of the magnesium and calcium was molten in an induction vacuum furnace under protective Ar atmosphere. The solidification of the melt was done by lowering and placing the crucible down onto a cooling element in the form of an actively cooled copper plate. To ensure directional solidification and avoiding shrinkage cavities within the billet, only the crucible's bottom was brought into contact with the cooling plate. As next step, the billets were homogenized at 350° C. for 12 hours and solutionized at 450° C. for 8 hours, followed by quenching in water or with pressurized air. The billets were then machined into three pieces of equal length, pre-heated in a convection furnace to the extrusion temperature for 30 min and subjected to hot extrusion at a constant extrusion ratio of 75. Tab. 1 provides an overview of all realized compositions and extrusion parameters. After extrusion, the extruded billet was characterized by metallography, electron backscatter diffraction, transmission electron microcopy, hardness measurements and tensile tests. Tensile tests were performed according to ISO 6892-1 at a strain rate of 0.001 per second.

As indicated earlier, the quantity “wt %” refers to “weight of an alloying element or impurity element per total weight of the alloy expressed as a fraction of 100”.

TABLE 1
Nominal compositions and extrusion parameters
of various synthesized Mg-Ca alloys.

| | Nominal Ca | Extrusion | Extrusion |
|---|---|---|---|
| | content in | speed in | temperature in |
| Billet ID | wt % | mm/s | ° C. |
| A12-E05-B1 | 0.45 | 0.1 | 345 |
| A12-E05-B3 | 0.45 | 0.1 | 360 |
| A12-E07-B2 | 0.45 | 0.05 | 370 |
| A12-E10-B1 | 0.45 | 0.05 | 330 |
| A12-E10-B2 | 0.45 | 0.25 | 375 |
| A12-E10-B3 | 0.45 | 0.5 | 390 |
| A12-E11-B1 | 0.2 | 0.05 | 280 |
| A12-E11-B2 | 0.2 | 0.25 | 315 |
| A12-E11-B3 | 0.2 | 0.5 | 345 |
| A12-E12-B1 | 0.2 | 0.1 | 300 |
| A12-E12-B2 | 0.2 | 0.25 | 315 |
| A12-E12-B3 | 0.2 | 0.5 | 345 |
| A12-E13-B1 | 0.3 | 0.05 | 300 |
| A12-E13-B2 | 0.3 | 0.25 | 345 |
| A12-E13-B3 | 0.3 | 0.5 | 360 |
| A12-E14-B1 | 0.6 | 0.05 | 345 |
| A12-E14-B2 | 0.6 | 0.25 | 360 |
| A12-E14-B3 | 0.6 | 0.5 | 375 |
| A12-E15-B1 | 0.2 | 0.15 | 310 |
| A12-E15-B2 | 0.2 | 0.15 | 315 |
| A12-E15-B3 | 0.2 | 0.2 | 315 |
| A12-E16-B1 | 0.3 | 0.15 | 330 |
| A12-E16-B2 | 0.3 | 0.1 | 330 |
| A12-E16-B3 | 0.3 | 0.15 | 345 |
| A12-E20-B1 | 0.45 | 0.25 | 360 |
| A12-E20-B2 | 0.45 | 1 | 390 |
| A12-E20-B3 | 0.45 | 1 | 375 |
| A12-E21-B1 | 0.6 | 0.15 | 360 |
| A12-E21-B2 | 0.6 | 0.25 | 375 |
| A12-E21-B3 | 0.6 | 1 | 375 |
| A12-F02-B1 | 0.2 | 8 | 345 |
| A12-F02-B3 | 0.2 | 8 | 330 |
| A12-F03-B1 | 0.3 | 0.05 | 345 |
| A12-F03-B2 | 0.3 | 8 | 345 |
| A12-F03-B3 | 0.3 | 0.17 | 345 |

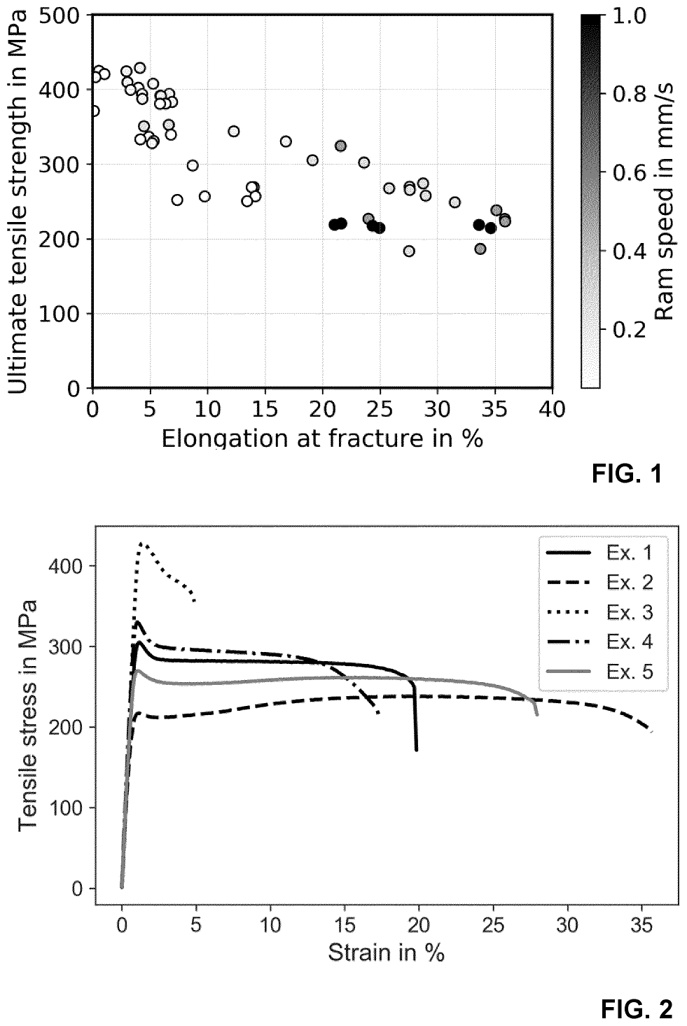

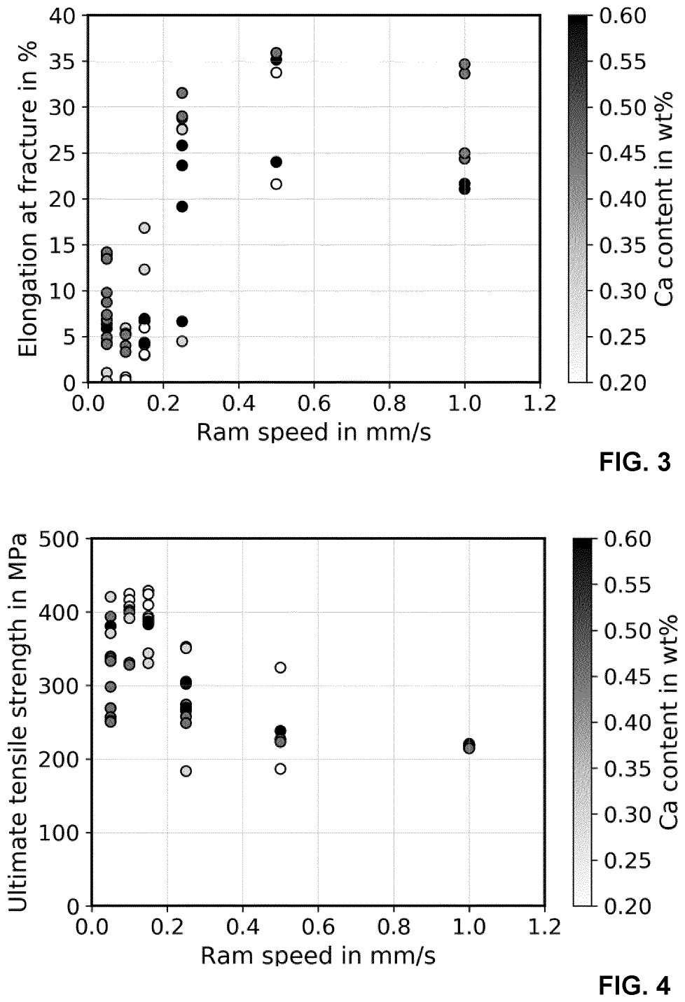

FIG. 1 shows the obtained results of ultimate tensile strength and elongation at fracture. Values of ultimate tensile strength of more than 430 MPa and values of elongation at fracture of more than 35% were achieved. The respective ram speeds are indicated in FIG. 1 in gradient gray levels, demonstrating a correlation of higher ram speeds with larger elongation at fracture (see also FIG. 3). FIG. 2 displays stress-strain-curves of deliberately chosen compositions and extrusion conditions to demonstrate the variety of tunable mechanical properties. The highest strength values were achieved for rather low contents of Ca and small ram speeds (FIG. 4).

TABLE 2
Billet identifications (ID) of displayed specimens in FIG. 2

| Figure legend | Billet ID |
|---|---|
| Ex. 1 | A12-E14-B2 |
| Ex. 2 | A12-E14-B3 |
| Ex. 3 | A12-E15-B2 |
| Ex. 4 | A12-E16-B3 |
| Ex. 5 | A12-E20-B1 |

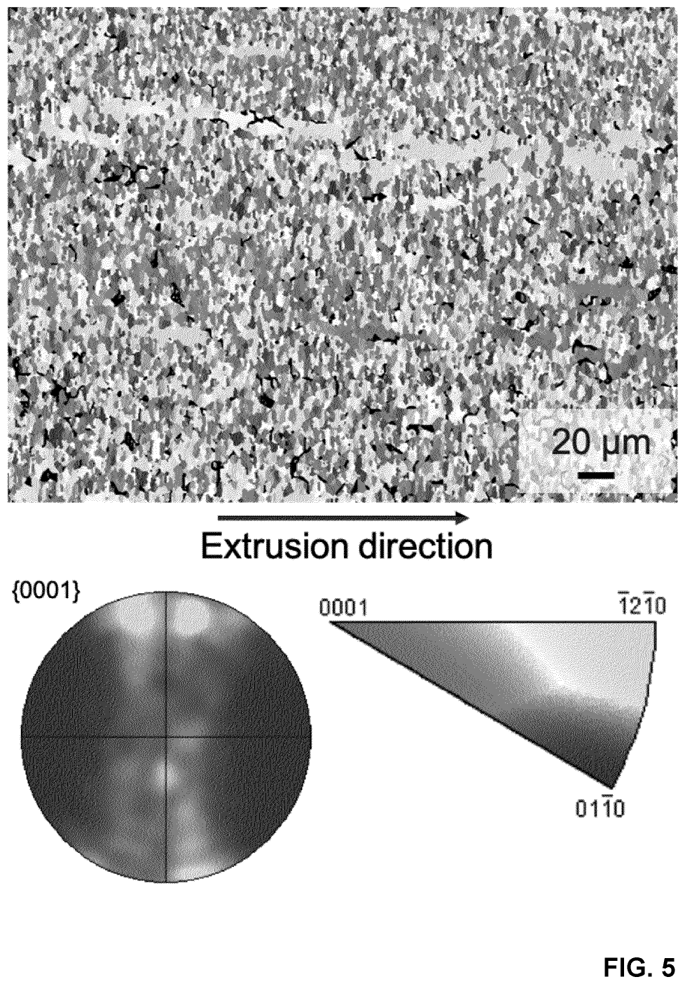

FIG. 5 shows the results from electron backscatter diffraction of an alloy that exhibited an ultimate tensile strength of 265 MPa at an elongation at fracture of 29%. The figure reveals a highly recrystallized microstructure and a pole figure exhibiting a wide angular distribution of the basal planes, a feature that is typically attributed to rare-earth element alloying additions and generally connected to exceptional strength and ductility of magnesium alloys. In this present embodiment this behavior is achieved solely by the alloying element Ca and only in a single hot-extrusion step.

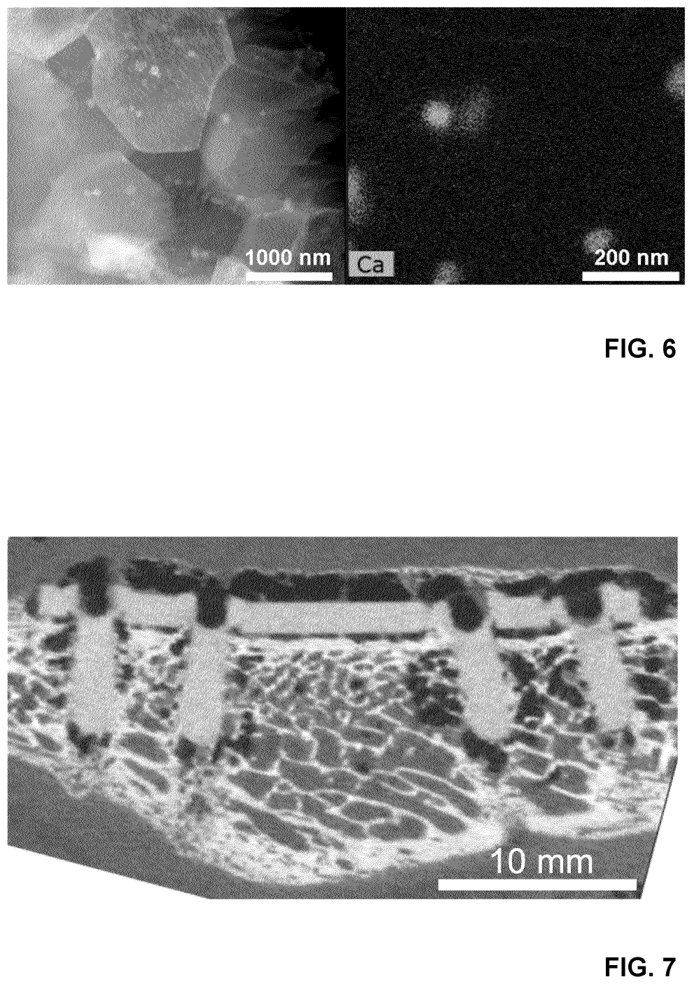

FIG. 6 shows an image as obtained by transmission electron microscopy, exhibiting grains of size of 1 micrometer and smaller, as well as finely distributed Mg₂Ca particles with a size of 100 nanometers or smaller. This alloy exhibits an ultimate tensile strength of more than 330 MPa at an elongation at fracture of more than 16%.

In general, a clear trend of larger grains at higher extrusion temperatures is observed. Moreover, an additional strong correlation with extrusion speed was found. Billets with the same chemical composition and extrusion temperature but extruded at different ram speeds exhibit larger grain size and consequently lower hardness and lower strength with increasing ram speed. Thus, only relatively moderate changes in extrusion speed can be used for optimization of the mechanical properties with respect to strength and ductility, while then too large ram speeds lead again to deterioration of the mechanical properties accompanied with enlarged grain size.

In general, a strain-rate softening behavior was observed: When keeping extrusion ratio, composition and extrusion temperature constant, the forces needed to perform the extrusion process according to the set parameters were found to decrease with increasing ram speed. Exemplarily, three alloys were extruded at an elevated ram speed of 8 mm/s (Tab. 1). They exhibited a tensile yield strength of less than 81 MPa and an ultimate tensile strength of less than 190 MPa at a mediocre elongation at fracture of about 23%. Post-extrusion investigations revealed that only very few Mg₂Ca intermetallic particles were present in those three alloys, contrary to other alloys extruded at the same extrusion temperature but ram speeds within the optimal range according to the invention.

Similarly, it was found that relatively small changes in extrusion temperature effect the mechanical properties of the extruded rod strongly. So, setting the processing window to achieve excellent and tunable mechanical properties in such alloys is not state-of-the-art, and represents a significant invention. Additionally, minute additions of preferably dissolved zirconium and/or hafnium can widen the process window to a technically easier controllable extent because of an additional grain-pinning and recrystallization-inhibition effect.

### In Vitro Corrosion Testing

First, a high-purity (99.5% pure) magnesium ingot was cut into smaller pieces with the aid of a band saw, and these pieces were machined subsequently to fit into a graphite crucible. Additionally, a hole was drilled into the top of the pieces to subsequently accommodate the calcium raw material pieces, with purity of 99% or higher. The assembly of the magnesium and calcium was molten in an induction vacuum furnace under protective Ar atmosphere. The solidification of the melt was done by lowering and placing the crucible down onto a cooling element in the form of an actively cooled copper plate. To ensure directional solidification and avoiding shrinkage cavities within the billet, only the crucible's bottom was brought into contact with the cooling plate. As next step, the billets were homogenized at 350° C. for 12 hours and solutionized at 450° C. for 8 hours, followed by quenching in water or with pressurized air. The billet was then pre-heated in a convection furnace to the extrusion temperature for 30 min and subjected to hot extrusion at a constant extrusion ratio of 75, a ram speed of 0.1 mm/s and an extrusion temperature of 360° C. The resulting material was subjected to in vitro corrosion tests in simulated body fluid in comparison to similarly prepared Mg—Zn—Ca alloys and extruded ultra-high purified Mg (total amount of impurities <0.001 wt %). The samples were immersed for 17 days at a controlled temperature of 37° C. and a CO₂-controlled pH of 7.4.

Tab. 3 provides the amount of salts introduced to 5 liters of deionized water to prepare the simulated body fluids.

TABLE 3
Ingredients and amount used to prepare 5 liters
of simulated body fluid based on deionized water.

| | Mass in |
|---|---|
| Ingredient | gram |
| KCl | 1.4913 |
| NaCl | 29.2200 |
| NaHCO₃ | 11.3426 |
| MgSO₄ | 1.2327 |
| CaCl₂ | 1.8378 |
| KH₂PO₄ | 0.6807 |

Tab. 4 provides an overview of the obtained degradation rates. The investigated Mg-0.45 wt % Ca exhibits degradation rates comparable to lean Mg—Zn—Ca alloys and ultrahigh-purified Mg.

TABLE 4
Degradation rates as measured in a simulated body fluid (SBF) immersion
test. Alloy compositions are provided in wt % (weight percent) and
comprise next to the provided amounts of alloying elements also
unavoidable impurities, with the balance being Mg. Ultrahigh-purified
Mg comprises a total amount of impurities <0.002 wt %.

| | | Standard |
|---|---|---|
| | Degradation rate in | deviation in |
| Alloy | mm/year | mm/year |
| Mg-1 wt % Zn-0.3 wt % Ca | 0.33 | 0.04 |
| Mg-0.45 wt % Zn-0.45 wt % Ca | 0.32 | 0.04 |
| Mg-0.2 wt % Zn-0.3 wt % Ca | 0.40 | 0.06 |
| Mg-0.45 wt % Ca | 0.34 | 0.02 |
| Ultrahigh-purified Mg | 0.33 | 0.02 |

### Biodegradable Implants

Further embodiments were synthesized in the form of biodegradable implantable screws and plates, and subjected to evaluation in a living sheep model for 8 weeks.

In total two billets were produced and subjected to hot extrusion.

First magnesium was purified by vacuum distillation to a purity higher than 99.99%. Subsequently, this ultra-high purified magnesium was cut into smaller pieces with the aid of a band saw, and these pieces were machined subsequently to fit into a graphite crucible. Additionally, a hole was drilled into the top of the pieces to subsequently accommodate the calcium raw material pieces in the amount 0.45% by weight based on the total weight of the alloy, with purity of 99% or higher. The assembly of the magnesium and calcium was molten in an induction vacuum furnace under protective Ar atmosphere. The solidification of the melt was done by lowering and placing the crucible down onto a cooling element in the form of an actively cooled copper plate. To ensure directional solidification and avoiding shrinkage cavities within the billet, only the crucible's bottom was brought into contact with the cooling plate. As next step, the billets were homogenized at 350° C. for 12 hours and solutionized at 450° C. for 8 hours, followed by quenching in water or with pressurized air. The billets were then pre-heated in a convection furnace to the extrusion temperature for 30 min and subjected to hot extrusion at a constant extrusion ratio of 75 (round cross-section) and 67 (rectangular cross-section), respectively. The billet of round cross-section was extruded at a ram speed of 0.05 mm/s and an extrusion temperature of 370° C., the billet of rectangular cross-section was extruded at 0.05 mm/s and 380° C.

From the extruded billet of round cross-section, screws of a thread diameter 2.7 mm were manufactured by conventional machining and from the extruded billet of rectangular cross-section, plates of the outer dimensions 31×6×1.6 mm³ were machined by milling. Half of the screws were subjected to a plasma electrolytic oxidation (PEO) surface treatment, whereas the other half remained untreated. The coating layer as produced by the PEO surface treatment was measured to have a thickness between 3 and 12 micrometers.

After gamma sterilization, the plates were implanted in sheep, two plates on the right pelvis and two on the left, using four screws each. Overall, seven surface-coated plates and seven untreated plates with the accompanying screws were implanted. After 8 weeks, the sheep were sacrificed. The implants with the surrounding tissue were then extracted and subjected to micro-computer tomography, mass loss measurements and histological investigations.

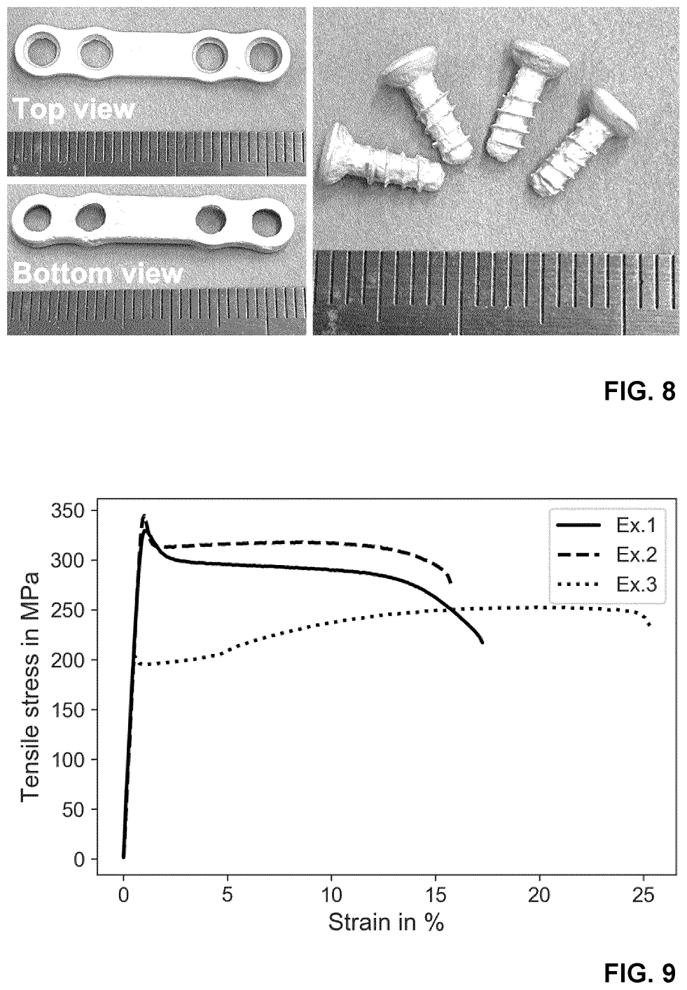

FIG. 7 shows a sectional image from micro-computer tomography of a Mg-0.45 wt % Ca implant (additionally to the provided Ca content the alloy comprises unavoidable impurities, with the balance being Mg) after 8 weeks of implantation time. FIG. 8 shows photographs of a Mg-0.45 wt % Ca plate and Mg-0.45 wt % Ca screws (additionally to the provided Ca content the alloy comprises unavoidable impurities, with the balance being Mg) extracted after 8 weeks of implantation time and chemical removal of the corrosion products. Screws and plate bottom (in contact with bone tissue) show clear visual signs of degradation, whereas the plate's top appears as being almost without signs of degradation.

Mass-loss measurements revealed an average degradation rate of 0.30 mm/year for the untreated implants and an average degradation rate of 0.28 mm/year for the PEO surface-treated implants. An assessment of soft tissue harvested in the vicinity of the plates revealed usual signs of chronic inflammation and fibrous tissue, but also enhanced ossification in all cases. No signs of potential biocompatibility intolerance were detected.

### Zr Additions

As further examples alloys nominally comprising 0-1 wt % Zn, 0.3-0.45 wt % Ca and 0-0.3 wt % Zr (detailed alloying contents: Tab. 5 and Tab. 6), with the remainder Mg and unavoidable impurities were prepared, Zr was introduced by employing a Mg—Zr master alloy of 30 wt % Zr. After melting and solidification of the alloy, the resulting billets were homogenized at 350° C. for 12 hours and subsequently at 450° C. for 8 hours, followed by quenching in water. Then, the billets were pre-heated in a convection furnace to the extrusion temperature for 30 min and subjected to hot extrusion at an extrusion ratio of 75 at different extrusion temperatures and ram speeds (see Tab. 5). It was found that the billets feature significantly different grain sizes after the last homogenization step, with the alloys containing Zr exhibiting significantly smaller grain sizes than the alloys without Zr (average grain size of tens of micrometers and several millimeters, respectively). A higher Zr-content clearly leads to finer grain size. This can be attributed to the well-known grain-refining effect of Zr during solidification of the magnesium alloy melt. Smaller initial grain size is desired because grain boundaries act as nucleation sites for dynamic recrystallization during the hot-extrusion process, and a smaller grain size in the billet before extrusion means a higher amount of grain boundaries, and consequently more options for recrystallized grains to nucleate.

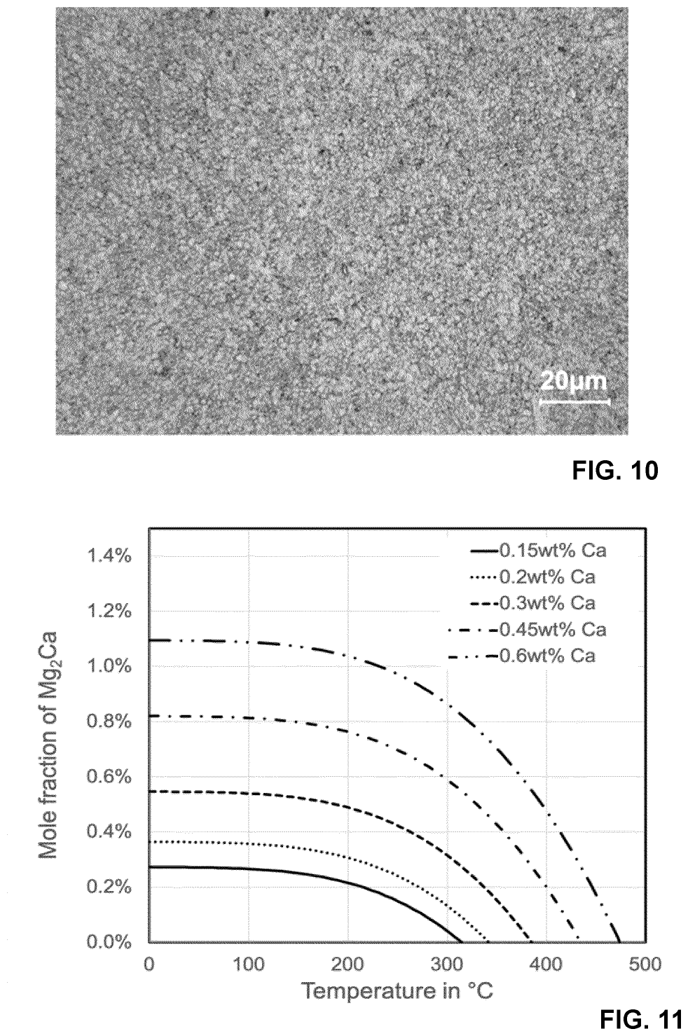

Furthermore, it was found that one tested Mg—Ca—Zn—Zr alloy can be extruded at temperatures of about 30° C. higher to achieve mechanical properties very comparable to the Mg—Ca alloy without Zr (see FIG. 9), with fully recrystallized microstructure (FIG. 10—Mg—Zn—Ca—Zr extruded at 375° C. and at a ram speed of 0.2 mm/s). The sometimes in literature and prior art described detrimental effect of Zr on the recrystallization during hot deformation can therefore not be validated. Quite contrary, it is shown that the smaller initial grain size and the possibility of elevated extrusion temperatures add to a fully recrystallized, but still fine-grained microstructure. The apparently contradicting results can be explained by the fact that alloys as described in literature typically feature amounts of Zr that is approximately 5 to 10 times larger than the additions described here. Also, all known literature sources on that matter refer to extrusion at a lower extrusion ratio. The stronger plastic deformations at the higher extrusion ratios as used in the present invention are expected to contribute to the observed excellent results.

Extrusion of the Mg—Ca—Zn—Zr and Mg—Ca—Zr alloys at even higher temperature (Tab. 5, Tab. 6 and FIG. 9) results in strength and elongation at fracture quite similar to the above-described extruded alloys without Zr.

Chemical analysis was performed on the extruded materials, which revealed some losses of Ca and Zr with respect to the nominal alloying contents (Tab. 6). This is related to the alloy processing and can be easily compensated when very precise compositions are needed.

In summary, the addition of minute amounts of Zr (nominally up to 0.3 wt %) was found to be very beneficial as it extends the possible process window towards higher temperatures and therefore the detected high sensitivity on process parameters with respect to Mg—Ca alloys can be significantly dimished.

It should be noted here that the Mg—Ca—Zn—Zr alloys were produced according to the method of the invention. These alloys however are not favorable as implantable medical devices because of the presence of zinc as alloying element. In fact, and as mentioned earlier, the presence of zinc in implants has revealed an increased degradation rate and thus generated an undesired high hydrogen release rate. Nevertheless, these examples clearly show that an alloy comprising magnesium and calcium and further metals such as zirconium, hafnium and zinc can be produced according to the method of the invention and result in extraordinary mechanical properties as well. In particular, these extraordinary properties can also be achieved with Mg—Ca—Zr alloys (without the addition of Zn), as can be verified with the Mg—Ca—Zr alloys provided in Tab. 5 and Tab. 6. It is to be emphasized that the realization of similar or better mechanical properties with Mg—Ca—Zr(—Hf) alloys (compared to Mg—Ca), due to the realization of a smaller grain size by the addition of Zr (and/or Hf) (see the Hall-Petch relation described above) and the concurrent substantial reduction of the processing sensitivity with the addition of Zr (and Hf), represents a major and significant result of the current invention.

TABLE 5
Composition and extrusion parameters of a lean Mg-Ca alloy and lean Mg-Ca
with Zn and Zr additions. Stress-strain curves according to ISO 6892-1 (strain
rate = 0.001 per second) of three examples are shown in FIG. 9 (A12-
Fx-1 labelled in FIG. 9 as “Ex. 1”; A12-Fx-2 labelled in FIG. 9 as “Ex.
2” and A12-Fx-3 labelled in FIG. 9 as “Ex. 3”). Alloy compositions
are provided in wt % (weight percent) and comprise next to the provided amounts
of alloying elements also unavoidable impurities, with the balance being Mg.

| | Nominal | Nominal | Nominal | | |
|---|---|---|---|---|---|
| | Ca | Zn | Zr | Extrusion | Extrusion |
| | content | content in | content | temperature | speed in |
| Identification | in wt % | wt % | in wt % | in ° C. | mm/s |
| A12-Fx-1 | 0.3 | — | — | 345 | 0.15 |
| A12-Fx-2 | 0.3 | 1.0 | 0.07 | 375 | 0.2 |
| A12-Fx-3 | 0.3 | 1.0 | 0.07 | 415 | 0.2 |
| A12-F16-B4 | 0.45 | — | 0.07 | 370 | 0.05 |
| A12-F17-B4 | 0.45 | — | 0.015 | 370 | 0.05 |
| A12-F18-B4 | 0.45 | — | 0.1 | 370 | 0.05 |
| A12-F19-B4 | 0.45 | — | 0.03 | 370 | 0.05 |
| A12-F21-B4 | 0.45 | — | 0.3 | 370 | 0.05 |
| A12-F16-B3 | 0.45 | — | 0.07 | 375 | 0.25 |
| A12-F17-B3 | 0.45 | — | 0.015 | 375 | 0.25 |
| A12-F18-B3 | 0.45 | — | 0.1 | 375 | 0.25 |
| A12-F19-B3 | 0.45 | — | 0.03 | 375 | 0.25 |
| A12-F21-B3 | 0.45 | — | 0.3 | 375 | 0.25 |
| A12-F22-B2 | 0.45 | — | 0.1 | 375 | 0.25 |
| A12-F22-B3 | 0.45 | — | 0.1 | 375 | 0.25 |
| A12-F16-B2 | 0.45 | — | 0.07 | 390 | 0.5 |
| A12-F17-B2 | 0.45 | — | 0.015 | 390 | 0.5 |
| A12-F18-B2 | 0.45 | — | 0.1 | 390 | 0.5 |
| A12-F19-B2 | 0.45 | — | 0.03 | 390 | 0.5 |
| A12-F21-B2 | 0.45 | — | 0.3 | 390 | 0.5 |

TABLE 6
Results on extruded lean Mg-Ca-Zr alloys. Ca and Zr content
were measured with Inductively Coupled Plasma Optical Emission
spectroscopy. Alloy compositions are provided in wt % (weight
percent) and comprise next to the provided amounts of alloying
elements also unavoidable impurities, with the balance being
Mg. Tensile parameters were determined according to ISO
6892-1 (strain rate = 0.001 per second).

| | | | Ultimate | |
|---|---|---|---|---|
| | Measured Ca | Measured Zr | tensile | |
| | content in | content in | strength in | Elongation at |
| Identification | wt % | wt % | MPa | fracture in % |
| A12-F16-B4 | 0.384 | 0.049 | 342.6 | 7.2 |
| A12-F17-B4 | 0.403 | 0.0047 | 305.4 | 7.9 |
| A12-F18-B4 | 0.409 | 0.071 | 344.6 | 7.9 |
| A12-F19-B4 | 0.397 | 0.0122 | 333.85 | 7.1 |
| A12-F21-B4 | 0.413 | 0.155 | 335.4 | 9.0 |
| A12-F16-B3 | 0.388 | 0.049 | 296.3 | 12.6 |
| A12-F17-B3 | 0.403 | 0.0056 | 241.95 | 22.5 |
| A12-F18-B3 | 0.399 | 0.071 | 264.2 | 19.0 |
| A12-F19-B3 | 0.388 | 0.0124 | 285.55 | 15.5 |
| A12-F21-B3 | 0.413 | 0.145 | 287.85 | 15.0 |
| A12-F22-B2 | 0.403 | 0.055 | 260.36 | 21.1 |
| A12-F22-B3 | 0.404 | 0.064 | 249.99 | 22.3 |
| A12-F16-B2 | 0.396 | 0.04 | 231.8 | 22.8 |
| A12-F17-B2 | 0.397 | 0.0033 | 211.95 | 26.3 |
| A12-F18-B2 | 0.39 | 0.06 | 226.25 | 26.0 |
| A12-F19-B2 | 0.382 | 0.009 | 229.55 | 25.7 |
| A12-F21-B2 | 0.399 | 0.129 | 233.1 | 25.4 |

1. A method of producing an alloy comprising magnesium and calcium, the method comprising the steps of:
generating a billet comprising magnesium and calcium; and
extruding the billet,
wherein the billet is extruded at least once at an extrusion temperature in the range of 250° C. to 450° C. and at a ram speed in the range of 0.01 mm/s to 1 mm/s and at an extrusion ratio in the range of 20 to 150.

2. The method according to claim 1, wherein the billet furthermore comprises zirconium and/or hafnium.

3. The method according to claim 1, wherein the alloy comprises less than 0.01% by weight of zinc based on the total weight of the alloy.

4. The method according to claim 1, wherein the alloy comprises between 0.15% by weight and 1.0% by weight of calcium based on the total weight of the alloy, and/or
wherein the alloy comprises 0.5% by weight or less of zirconium based on the total weight of the alloy and/or wherein the alloy comprises between 0.005% zirconium by weight and 0.5% zirconium by weight, and/or
wherein the alloy comprises 0.5% by weight or less of hafnium based on the total weight of the alloy, and/or wherein the alloy comprises between 0.005% hafnium by weight and 0.5% hafnium by weight, and/or
wherein a remainder comprises magnesium and possibly additionally impurities.

5. The method according to claim 1, wherein the magnesium and/or the calcium is purified by vacuum distillation prior to the generation of the billet, and/or
wherein the billet is generated by vacuum distillation.

6. The method according to claim 1, wherein the billet is generated by melting calcium and magnesium, whereby a melt comprising calcium and magnesium is formed, and by subsequently solidifying the melt, and
wherein zirconium is provided in the form of a magnesium-zirconium master alloy and/or wherein hafnium is provided in the form of a magnesium-hafnium master alloy.

7. The method according to claim 6, wherein a melting temperature is in the range of 650° C. to 900° C., and/or
wherein a melting temperature is maintained for a time period, and/or
wherein the melt is formed by inductive heating, and/or
wherein the melt is stirred by currents being induced by inductive heating and/or by mechanical stirring and/or by ultrasonic waves.

8. The method according to claim 6, wherein the melt is solidified by generating the melt in a crucible and subsequent casting, or wherein the melt is solidified by generating the melt in a crucible and by arranging the crucible subsequently on a cooling element,
wherein the cooling element is a block of material, and/or the cooling element being actively cooled.

9. The method according to claim 1, further comprising at least one step of homogenization annealing heat treatment being performed after the billet is generated and before the billet is extruded, and
wherein the calcium and additionally the zirconium and/or the hafnium are brought into solid solution, and/or
wherein an annealing temperature is in the range of 300° C. to 520° C. and/or wherein a holding period is 0.5 hours or more, and/or
wherein two or more annealing steps are performed at successively increasing annealing temperature.

10. The method according to claim 1, wherein the billet is preheated prior to the extrusion, and/or
wherein Mg₂Ca intermetallic particles are formed prior to the extrusion, the Mg₂Ca intermetallic particles having a size of 500 nanometers or less.

11. The method according to claim 1, further comprising the step of performing a heat treatment after the extrusion of the billet, the heat treatment being performed at a temperature in the range of 150° C. to 350° C. and/or with a holding period of 30 seconds or more, and/or
further comprising the step of coating at least part of the alloy, the coating being a plasma electrolytic anodization coating and/or an amorphous metallic coating and/or a fluoric conversion coating and/or a Mg(OH)₂ coating and/or a calcium phosphate conversion coating and/or a hydroxy-apatite coating and/or an organic coating and/or a biodegradable polymer coating and/or a sol-gel coating.

12. An alloy comprising magnesium and calcium, an alloy as produced in claim 1,
wherein the alloy has an ultimate tensile strength in the range of 100 MPa to 500 MPa and an elongation at fracture in the range of 2% to 50%.

13. The alloy according to claim 12, wherein the alloy comprises between 0.15% by weight and 1.0% by weight of calcium based on the total weight of the alloy, and/or
wherein the alloy comprises 0.1% by weight or less of zirconium based on the total weight of the alloy and/or wherein the alloy comprises between 0.005% zirconium by weight and 0.1% zirconium by weight, and/or
wherein the alloy comprises 0.1% by weight or less of hafnium based on the total weight of the alloy, and/or wherein the alloy comprises between 0.005% hafnium by weight and 0.1% hafnium by weight, and/or
wherein a remainder comprises magnesium and possibly additionally impurities.

14. The alloy according to claim 12, wherein the alloy comprises Mg₂Ca intermetallic particles, the Mg₂Ca intermetallic particles having a size of 500 nanometers or less, and/or
wherein the alloy forms a fine-grained structure with an average grain size of 5 micrometers or less, the Mg₂Ca intermetallic particles being distributed dispersedly at grain boundaries of the fine-grained structure and/or in grains of the fine-grained structure, and/or
wherein the alloy further comprises at least partially a coating, and/or
wherein the alloy has a degradation rate being smaller than 1 millimeter per year according to the testing standard ASTM F3268.

15. A method comprising producing an implantable medical device comprising the alloy according to claim 12, the implantable medical device being an implant and/or being biodegradable and/or configured to be a tool in orthopedic surgery and/or in dental applications and/or in vascular intervention and/or in veterinary medicine.

16. An implantable medical device comprising or consisting of an alloy according to claim 12, the implantable medical device being an implant and/or being biodegradable and/or being configured to be a tool in orthopedic surgery and/or in dental applications and/or in vascular intervention and/or in veterinary medicine.
